(19) United States
(12) Patent Application Publication
(10) Pub. No.: US 2024/0021313 A1
(43) Pub. Date: Jan. 18, 2024

(54) SYSTEM AND A METHOD FOR DETECTING AND QUANTIFYING ELECTROENCEPHALOGRAPHIC BIOMARKERS IN ALZHEIMER'S DISEASE

(71) Applicant: Beacon Biosignals, Inc., Boston, MA (US)

(72) Inventors: Matthew S. Alkaitis, Redwood City, CA (US); Jarrett Revels, Brooklyn, NY (US); Jacob A. Donoghue, Boston, MA (US); Jay Srirarm Pathmanathan, Wellesley, MA (US)

(73) Assignee: Beacon Biosignals, Inc., Boston, MA (US)

(21) Appl. No.: 18/358,333

(22) Filed: Jul. 25, 2023

Related U.S. Application Data
(63) Continuation-in-part of application No. 17/863,803, filed on Jul. 13, 2022.
(60) Provisional application No. 63/392,333, filed on Jul. 26, 2022.
(60) Provisional application No. 63/221,746, filed on Jul. 14, 2021.

(51) Int. Cl. G16H 50/20; G16H 10/20; A61B 5/00; A61B 5/372

(52) U.S. Cl. CPC G16H 50/20; G16H 10/20; A61B 5/4088; A61B 5/372; A61B 5/7267; A61B 5/4094

(57) ABSTRACT

The present disclosure relates to a system and method capable of capturing, processing and analyzing electroencephalography (EEG) signals to deliver or support an expert interpreter in delivering a clinically-relevant interpretation for patients with Alzheimer's disease. Such interpretations include identification of diagnostic, prognostic or risk stratification neurobiomarkers to evaluate subjects for suitability in clinical trials, monitor for clinical conditions, detect adverse effects of interventions, identify efficacy of interventions or identify subjects that are more or less likely to respond to therapeutic interventions in a specified manner.

### RELATED APPLICATIONS

This Application is a Non-Provisional of Provisional (35 USC 119(e)) of U.S. Application Ser. No. 63/392,333, filed Jul. 26, 2022, entitled “SYSTEM AND A METHOD FOR DETECTING AND QUANTIFYING ELECTROENCEPHALOGRAPHIC BIOMARKERS IN ALZHEIMER'S DISEASE” and this Application is a Continuation-in-part of U.S. application Ser. No. 17/863,803, filed Jul. 13, 2022, entitled “SYSTEMS AND METHODS FOR RAPID NEUROLOGICAL ASSESSMENT OF CLINICAL TRIAL PATIENTS”, which is a Non-Provisional of Provisional (35 USC 119(e)) of U.S. Application Ser. No. 63/221,746, filed Jul. 14, 2021, entitled “SYSTEMS AND METHODS FOR RAPID NEUROLOGICAL ASSESSMENT OF CLINICAL TRIAL PATIENTS”, each of these applications is herein incorporated by reference in its entirety.

### BACKGROUND OF INVENTION

The applicants appreciate that Alzheimer's is a complex disease, progressing over many years, with limited treatment options. In this context improved neurophysiological assessments could accelerate basic understanding of Alzheimer's disease, accelerate development of novel therapeutics, monitor patients receiving treatment, stratify patient populations or function as a companion diagnostic for treatment selection. In some instances, it may be challenging to statistically observe a therapeutic effect unless a smaller subset of the study population is selected. It may also be the case that specific therapeutic mechanisms may target subsets of Alzheimer's patients with specific disease features, physiology or likelihood of progression. For these instances, additional data may be collected using a “companion diagnostic” to help stratify patients by their likelihood of responding to a given therapy. Companion diagnostics may also be used to stratify patients by their likelihood of experiencing an adverse event, which helps clinicians select which patients should not receive a given therapy.

One of the most challenging types of data to collect and interpret is electroencephalography (EEG) data. EEG is a method of capturing the electrical impulses generated by the nervous system via sensing devices (‘electrodes’ or other technologies) that are placed on the scalp. EEG signals are useful for the assessment of patients with Alzheimer's disease not only in clinical trials, but also clinically as a diagnostic assessment of typical or atypical neural function. For example, it is increasingly recognized that subsets of patients with Alzheimer's disease demonstrate overt epileptiform seizures with significant epileptiform discharges present between seizure events. Other patients have been noted to have epileptiform discharges in the absence of clinical seizures. Assessment of electrophysiologic biomarkers to identify such patient segments would aid in the design of targeted clinical trials, to develop improved prognostic assessments or as an indication for therapy.

The collection and analysis of EEG signals and other biomarkers associated with EEG data collection requires specialized equipment, skilled technicians and medical experts to perform data acquisition and interpretation. This standard approach often limits both the clinical utility of EEG and its utility in clinical trials due to extended technical set-up time, ongoing monitoring of the state of the system by trained experts, significant time and expense to arrange for expert interpretation, and delays in returning final interpretations to a study sponsor or the primary healthcare provider responsible for the care of a patient to make any relevant clinical decision(s). Furthermore, certain electrophysiologic biomarkers may be identified by statistical models that would not otherwise represent standard signatures recognized by trained experts. Standard clinical workflows also require medical experts to spend significant time scanning through large volumes of EEG data with frequent non-diagnostic artifacts in order to identify clinically-relevant signals.

### SUMMARY OF INVENTION

The applicants appreciate that EEG interpretation methods and apparatuses are not optimal in their ease of use, ability to handle large volumes of EEG data, ability to deliver rapid turn-around reporting, ability to identify novel electrophysiologic signatures or ability to be implemented as a companion diagnostic, among other issues. In particular, it is appreciated that clinical trials designed to evaluate novel medical devices, pharmaceutical interventions or other therapeutic interventions may benefit from EEG-based methods to determine trial eligibility, stratify patient populations, detect adverse events, identify endpoints or otherwise monitor trial participants for assessments at a given time point or over a period of time. It is also appreciated that human clinicians often demonstrate variability in interpretation of the same raw EEG data, where obtaining expert consensus is time-consuming and requires additional investment. In many cases, standard-of-care clinical interpretations are delivered without the clinician having read every data point available from the study. Given the volume of data generated by routine EEG assessment, it is almost impossible for a clinician to manipulate the data into multiple montage perspectives for all data points, even though particular montage displays may be the key view to make the diagnosis.

Some conventional systems provide for automated interpretation of EEG signals. However, the wide majority of these systems are implemented for the purposes of direct clinical intervention, such as modulation of an implanted electrostimulation device. In contrast, in some embodiments described herein, a system and method is optimized for the use of EEG signal detection and interpretation of electrophysiologic signatures in Alzheimer's disease patients. It should be appreciated that the implementation of such a system in the setting of a clinical trial for a novel therapeutic could also provide high-quality clinical data to support subsequent implementation more broadly in relevant clinical paradigms.

Some embodiments described herein include systems and methods comprising an EEG examination device, a display device, cloud-enabled processing and data accessibility, and a distributed computing environment that can be shared amongst one or more of the EEG examination device, the display device, and/or a remote computing device implemented for the purposes of assessing patients with Alzheimer's disease. In one embodiment the EEG examination device is a traditional EEG examination device commonly available for clinical or research use. In another embodiment, said EEG examination device is a low-profile head-mounted wireless recording device that may be rapidly applied to the individual being examined.

Data collected by the EEG examination device is ingested into an analytics system which is capable of identifying features, signatures or patterns that have significance for diagnosis, prognosis, risk-stratification or other medically-pertinent observations, estimates or predictions. The analytics system may employ a distributed computing environment in which computing is distributed across a traditional EEG examination device, a local computing device, head-mounted recording device, a display device, a remote computing device, or some combination thereof. This analysis may be performed with or without the use of other additional data such as electronic medical record data, ingestion of other data streams (e.g., heart rate, temperature or pulse oximetry) and/or a corpus of previously collected EEG data. A display device enables the operator to provide input to set the parameters of the exam, if required. The same or a different display device may provide real-time data feeds or summary analysis to an expert and/or a healthcare provider responsible for care of the subject and/or the subject themselves for interpretation. In some embodiments, a display device provides data to a clinician for interpretation. In other embodiments, some or all interpretation may be performed through the use of said analytics system, which may employ statistical modeling, machine learning algorithms, or other mathematical interpretation. Said statistical or machine learning models may be trained, stored and implemented to perform a range of analysis tasks. A machine learning or statistical model may be implemented to perform one seven exemplary tasks: 1) Identify patients with Alzheimer's disease and/or discriminate between patients with Alzheimer's disease and other neurodegenerative diseases such as frontotemporal dementia or Lewy Body dementia, 2) identify or predict patient subgroups with regard to neurophysiology, likelihood of cognitive decline or other clinically-relevant grouping, 3) screen patients prior to enrollment for inclusion or exclusion criteria, 4) identify or predict correlations with other clinically-relevant biomarkers such as protein-based biomarkers or imaging features, or clinical assessments such as cognitive scoring systems, 5) identify indication for therapeutic intervention, 6) identify signals associated with clinical benefit or predict clinical benefit following exposure to one or more therapies, and/or 7) identify signals associated with adverse events or predict adverse events following exposure to one or more therapies. Said methods may optionally include comparison to a disease-free, healthy normal distribution or other relevant data set. The analytics system may perform fully-automated interpretation or generate virtual guide elements to aid a human expert perform the interpretation. The system is also capable of generating comprehensive static or dynamic reports describing the results of relevant analyses that have been performed with or without confirmation by a licensed practitioner. In one embodiment, said system may employ data collection, analysis, and reporting in real-time. In another embodiment, one or a combination of these actions may be performed asynchronously with respect to data collection.

Further, various embodiments described herein may be performed alone or in combination with any of the embodiments described in U.S. patent application Ser. No. 17/863,803 entitled “SYSTEMS AND METHODS FOR RAPID NEUROLOGICAL ASSESSMENT OF CLINICAL TRIAL PATIENTS” filed Jul. 13, 2022, incorporated by reference by its entirety.

According to one aspect an electroencephalography (EEG) processing system for training one or more statistical models to analyze neurophysiology associated with Alzheimer's disease is provided. The system comprises an EEG detector device comprising an array of sensors, one or more processors, a computer-readable storage media coupled to the one or more processors, and an analysis pipeline implemented by the one or more processor configured to ingest and process electrical signals from the EEG detector device, wherein said one or more computer-readable storage media are further configured to store processor-executable instructions that, when executed by the at least one computer hardware processor, cause the at least one computer hardware processor to perform: retrieving a trained statistical model from at least one storage device, wherein the trained statistical model is trained on a plurality of annotated EEG data, wherein the plurality of EEG training data includes at least one annotation describing an entity of interest selected from a group comprising: specific dementia diagnosis, cognitive scores, behavioral scores, rate of cognitive decline, survival time, drug response, patient level phenotype, genetic mutations, protein biomarkers, imaging biomarkers, likelihood of adverse reaction, inclusion or exclusion criteria for a clinical trial, processing, using the first trained statistical model, EEG data from a subject to generate relevant output labels for said entity of interest; and storing the predicted entity of interest on the at least one storage device.

According to one embodiment said one or more computer-readable storage media are further configured to store processor-executable instructions that, when executed by the at least one computer hardware processor, cause the at least one computer hardware processor to perform: retrieving a first trained statistical model from at least one storage device, wherein the trained statistical model is trained on a plurality of annotated EEG data, wherein the plurality of EEG training data includes at least one annotation describing EEG features or signatures for at least one segment of EEG waveforms, processing, using the first trained statistical model, EEG data from a subject to generate relevant output labels for EEG features or signatures, extracting values for one or more features or signatures from the EEG data annotated by the first trained statistical model, retrieving a second trained statistical model from the at least one storage device, wherein the second trained statistical model is trained on extracted values for the one or more features from the plurality of annotated EEG data, processing, using the second trained statistical model, the values for the one or more features extracted from the annotated EEG data to predict an entity of interest from said group of entities of claim 1, and storing the predicted entity of interest on the at least one storage device.

According to one embodiment said EEG features or signatures are one or more from the group comprising: epileptiform discharges, seizures, power spectral frequencies, small sharp spikes, and sleep spindles. According to one embodiment said values for one or more features or signatures are one or more from the group comprising: number of epileptiform discharges, rate of epileptiform discharges, topographic distribution of epileptiform discharges, amplitude of epileptiform discharges, number of seizures, duration of seizures, changes in power spectral frequency distributions, number of small sharp spikes, rate of small sharp spikes, topographic distribution of small sharp spikes, amplitude of small sharp spikes, number of sleep spindles, rate of sleep spindles, and topographic distribution of sleep spindles.

According to one embodiment said prediction of said entity of interest is provided to a clinician as a companion diagnostic as an indication for a therapy for Alzheimer's disease. According to one embodiment at least one of the first or second trained statistical model is a convolutional neural net. According to one embodiment at least one of the first or second trained statistical models include a generalized linear model, a random forest, a support vector machine, and/or a gradient boosted tree.

According to one aspect an electroencephalography (EEG) processing system for training one or more statistical models to analyze neurophysiology associated with Alzheimer's disease is provided. The system comprises an EEG detector device comprising an array of sensors, one or more processors, a computer-readable storage media coupled to the one or more processors, and an analysis pipeline implemented by the one or more processor configured to ingest and process electrical signals from the EEG detector device, wherein said one or more computer-readable storage media are further configured to store processor-executable instructions that, when executed by the at least one computer hardware processor, cause the at least one computer hardware processor to perform: accessing a plurality of training annotated EEG recordings associated with a group of patients in a randomized controlled clinical trial or an observational study, wherein each of the plurality of training annotated EEG recordings is associated with diagnosis data for a respective patient, wherein each of the plurality of training EEG recordings includes at least one annotation describing an EEG feature signature or characteristic category for a portion of the recording, wherein the plurality of training annotated EEG recordings includes: a first plurality of annotated EEG recordings associated with a first group of patients with Alzheimer's disease, and a second plurality of annotated EEG recordings associated with a second group of patients belonging to a control group without Alzheimer's disease or with a specific alternate diagnosis, training one or more statistical models based on said plurality of training EEG data and said plurality of annotations, storing the one or more trained models on at least one storage device, and processing, using the one or more trained models, EEG data to predict the diagnosis of a new individual or new group of patients not previously used to train said one or more statistical models.

According to one embodiment said plurality of annotated training EEG recordings includes: a first plurality of annotated EEG recordings associated with a first group of patients with Alzheimer's disease with accelerated cognitive decline, and a second plurality of annotated EEG recordings associated with a second group of patients belonging to a control group with Alzheimer's disease but without accelerated cognitive decline, and said computer-readable storage media are further configured to store processor-executable instructions that, when executed by the at least one computer hardware processor, cause the at least one computer hardware processor to process, using the one or more trained models, EEG data to predict accelerated cognitive decline in a new individual or new group of patients not previously used to train said one or more statistical models.

According to one embodiment said plurality of annotated training EEG recordings includes: a first plurality of annotated EEG recordings associated with a first group of patients with Alzheimer's disease that meet inclusion and exclusion criteria for a clinical trial, and a second plurality of annotated EEG recordings associated with a second group of patients belonging to a control group with Alzheimer's disease but do not meet inclusion and exclusion criteria for a clinical trial, and said computer-readable storage media are further configured to store processor-executable instructions that, when executed by the at least one computer hardware processor, cause the at least one computer hardware processor to process, using the one or more trained models, EEG data to predict whether a new individual or new group of patients not previously used to train said model meet inclusion and exclusion criteria for a clinical trial.

According to one embodiment said plurality of annotated training EEG recordings includes: a first plurality of annotated EEG recordings associated with a first group of patients with Alzheimer's disease that are treated with a therapy, and a second plurality of annotated EEG recordings associated with a second group of patients belonging to a control group with Alzheimer's disease but are not treated with the same therapy, and said computer-readable storage media are further configured to store processor-executable instructions that, when executed by the at least one computer hardware processor, cause the at least one computer hardware processor to process, using the one or more trained models, EEG data to predict whether a new individual or new group of patients not previously used to train said model is treated with said therapy.

According to one embodiment said plurality of annotated training EEG recordings includes: a first plurality of annotated EEG recordings associated with a first group of patients with Alzheimer's disease that are treated with a therapy, and a second plurality of annotated EEG recordings associated with a second group of patients belonging to a control group with Alzheimer's disease but are not treated with a different therapy, and said computer-readable storage media are further configured to store processor-executable instructions that, when executed by the at least one computer hardware processor, cause the at least one computer hardware processor to process, using the one or more trained models, EEG data to predict the clinical response of a new individual or new group of patients not previously used to train said model to either said therapy used to treat the first group of patients or said therapy used to treat the second group of patients.

According to one embodiment said plurality of annotated training EEG recordings includes: a first plurality of annotated EEG recordings associated with a first group of patients with Alzheimer's disease that are treated with a therapy, and a second plurality of annotated EEG recordings associated with a second group of patients belonging to a control group with Alzheimer's disease but are not treated with a different therapy, and said computer-readable storage media are further configured to store processor-executable instructions that, when executed by the at least one computer hardware processor, cause the at least one computer hardware processor to process, using the one or more trained models, EEG data to predict the likelihood of adverse events of a new individual or new group of patients not previously used to train said model to either said therapy used to treat the first group of patients or said therapy used to treat the second group of patients.

According to one aspect a method is provided. The method comprises receiving electroencephalography (EEG) signals associated with a subject, processing, by a statistical model, the EEG signals associated with the subject, and performing a determination, by the statistical model, of an annotation of the EEG signals associated with the subject relevant to neurophysiology associated with Alzheimer's disease.

According to one embodiment the annotation includes one or more of a group comprising: specific dementia diagnosis, cognitive scores, behavioral scores, rate of cognitive decline, survival time, drug response, patient level phenotype, genetic mutations, protein biomarkers, imaging biomarkers, likelihood of adverse reaction, and inclusion or exclusion criteria for a clinical trial.

According to one embodiment, the method further comprising an act of identifying, responsive to the determination, whether the subject has Alzheimer's disease. According to one embodiment, the method further comprising an act of identifying, responsive to the determination, whether the subject more likely has a neurogenerative disease other than Alzheimer's disease. According to one embodiment, the method further comprising an act of identifying, responsive to the determination, whether the subject should be categorized in a subgroup of subjects having similar neurophysiology. According to one embodiment, the method further comprising an act of identifying, responsive to the determination, whether the subject is predicted to have clinically relevant biomarkers, imaging features, or clinical assessments.

According to one embodiment, the method further comprising an act of identifying, responsive to the determination, whether the subject is indicated for a therapeutic intervention. According to one embodiment, the method further comprising an act of identifying, responsive to the determination, whether the subject is predicted to have a clinical benefit to one or more therapies. According to one embodiment, the method further comprising an act of identifying, responsive to the determination, whether the subject is predicted to have an adverse event responsive to receiving one or more therapies.

This summary is a simplified and condensed description of the underlying conceptual framework, enabling technology, and possible embodiments, which are further explained in the Detailed Description below. This Summary is not intended to provide a comprehensive description of essential features of the invention, nor is it intended to define the scope of the claimed subject matter.

It should be appreciated that all combinations of the foregoing concepts and additional concepts discussed in greater detail below (provided such concepts are not mutually inconsistent) are contemplated as being part of the inventive subject matter disclosed herein. In particular, any and all combinations of the subject matter (both claimed and not) are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale. In the figures:

FIG. 1A is a diagram depicting one exemplary set of components capable of implementing the system and method. In this example, the display device, remote processing module, electronic medical record, database(s) or data repositories, other detector system(s) and computing or analytics system(s) interact by means of a communication network or networks.

FIG. 1B is a diagram depicting one exemplary set of components capable of implementing the system and method. In this example, the display device, remote processing module, electronic medical record, database(s) or data repositories, other detector system(s) and computing or analytics system(s) interact by means of a communication network or networks.

FIG. 2 is a block diagram depicting an exemplary set of functional components, including embodiments of processing environment components capable of implementing various aspects of the technology.

FIG. 3 is a block diagram that depicts an embodiment of the software architecture that enables EEG signals to be communicated between the data-preprocessing engine, signal processing, capture and storage engine and data analysis engine.

FIG. 4A is a flow chart illustrating an embodiment of a method implemented by the system to accomplish the general goal of collecting appropriate data streams, analyzing said data and optionally generating a report describing the results of said analysis.

FIG. 4B is a flow chart showing detail of step 420 from FIG. 4A, in which necessary data inputs are collected and integrated;

FIG. 4C is a flow chart of showing detail of step 430 from FIG. 4A, in which exam signal quality is assessed.

FIG. 4D is a flow chart showing detail of step 440 from FIG. 4A, in which data streams are analyzed with or without assistance from machine learning statistical models or with optional user input in conjunction with machine learning statistical models.

FIG. 4E is a flow chart of showing detail of step 4420 from FIG. 4D, in which a baseline and/or “normal” comparator is established for the purposes of evaluating the current data streams against said baseline and/or “normal” comparator;

FIG. 4F is a flow chart showing a method for training a statistical model used in step 4424 from FIG. 4D;

FIG. 4G is a flow chart showing a method for implementing a statistical model in step 4424 from FIG. 4D in which data is ingested by a statistical model and clinical features, signatures, population-level stratification or other outputs are identified by said statistical model;

FIGS. 5A-I are examples of virtual guide elements that are used to identify features or signatures for the purpose of identifying diagnostically-relevant signals or patterns.

FIG. 6 is an exemplary embodiment of the display interface that would enable an expert to interrogate the data captured by the detection device with the assistance of virtual guide elements and the ability to annotate or confirm diagnostic interpretations.

FIG. 7 is an exemplary embodiment of a report that describes the results of the system's analysis with or without user input as generated by the method described in FIG. 4A.

FIG. 8A is an exemplary summary illustration of visual elements that are presented to the patient or examinee using the disclosed system and method.

FIG. 8B is an exemplary summary illustration of visual elements that are presented to the patient or examinee using the disclosed system and method.

FIG. 9 is an exemplary summary illustration of report elements that are presented to the examiner or physician using the disclosed system and method.

FIG. 10 is an exemplary summary illustration of the communication workflow of the disclosed system and method.

### DETAILED DESCRIPTION OF INVENTION

Some embodiments discussed herein comprise a system and method of obtaining, processing and analyzing an EEG signal in order to do one, two, or all of: simplifying the application of an EEG acquisition device, reducing examinee discomfort, improving the quality or quantity of the data acquired during the examination, reducing inter-operator variability, decreasing interpretation turn-around time, functioning as a qualified companion diagnostic and/or identifying features, signatures, patterns or characterizations that were previously difficult or impossible to reproducibly assess by qualified experts.

In some embodiments, a system is provided that includes a head-mounted EEG detector device, including an array of electrodes that adhere to the forehead, as well as zero, one, or a plurality of medical examination devices that include but are not limited to, pulse oximeters, temperature probes, accelerometers, gyroscopes etc. One or more components of the head-mounted device may be disposable. Such a system may also include a display device to gather input from the subject or the operator about zero, one, or more of the following: the exam being performed, specific symptoms the subject is experiencing, biometric data or other relevant medical or personal data. During an examination, the head-mounted EEG detection device ingests, processes and transmits EEG data and/or data from other sensors. The method by which local processing is performed may or may not be updated throughout the examination based on feedback from the local processor or via a communications network such as Bluetooth, Wi-Fi, or some other communication method or protocol.

At least some methods utilized herein include analysis of raw or processed EEG data to generate virtual guiding elements to guide review, annotation or interpretation of the EEG data by a non-expert or a qualified expert such as a medical doctor. These virtual guiding elements may include but are not limited to instruction in the form of plain text or otherwise, one or more arrows, targets, circles, color-changing elements, progress bars, transparent elements, angles, projections of real-time or stored EEG or sensor data, or other virtual items. These virtual items may be related to features or signatures that may be previously known to the field via publication, training, word-of-mouth or expert annotations. Alternatively, these virtual items may be related to novel features, signatures or elements that are extracted algorithmically from individual EEG exam data or from a corpus of previous or simultaneous EEG exams. Other embodiments of the system are capable of generating static or dynamic reports that describe clinical outputs of interest, such as likelihood of disease progression, presence or absence of specific features, identification of patient subgroups, indications for a given therapeutic, likelihood of response to one or more therapeutics or likelihood of an adverse effect from one or more therapeutics among other read-outs.

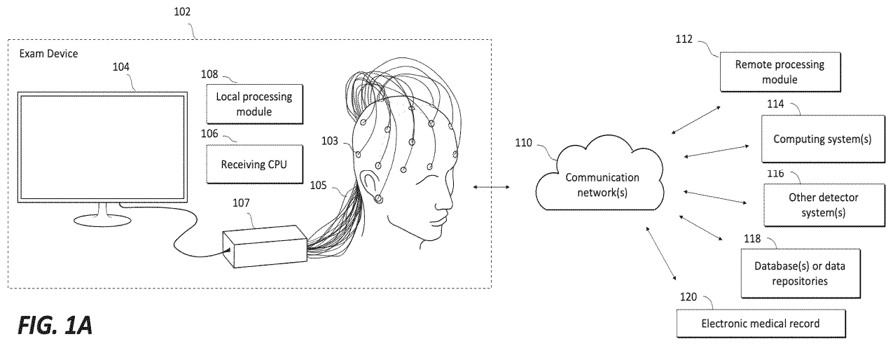

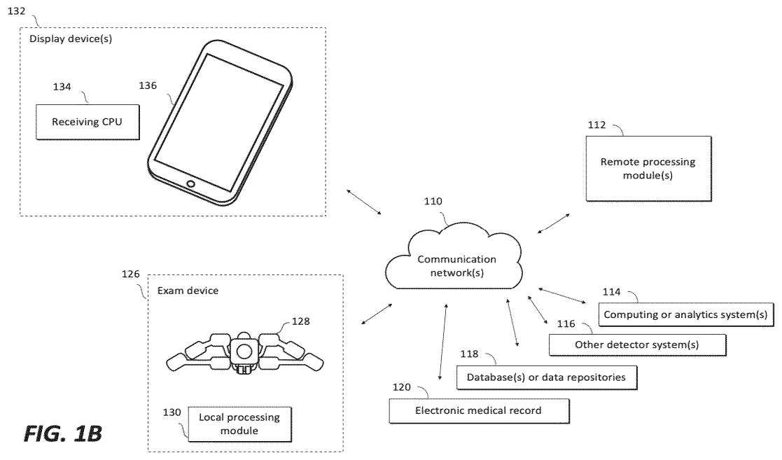

FIG. 1A depicts an exemplary set of hardware and functional components capable of implementing at least some embodiments described herein. In this example, a traditional EEG detector device and display system 102 includes a local processing module 108 that interacts with a communication network 110 or plurality of networks such as a Bluetooth or Wi-Fi to communicate with the remote processing module 112, electronic medical record 120, database(s) or data repositories 118, other detector system(s) 116 and computing or analytics system(s) 114.

To use this system, the examinee or an examiner, who may be a healthcare provider, technician or other operator would apply traditional EEG detection electrodes 103 to the examinee's head. FIG. 1A depicts a commonly employed 21-electrode lead placement arrangement familiar to one skilled in the art; however, in some embodiments fewer or more electrode leads may be deployed and placed in a plurality of different positions. EEG signal is captured by traditional EEG electrodes 103 and communicated via transmission cable 105 to local processing module 108. One, both, or neither of receiving CPU 106 and local processing module 108 may be housed in housing unit 107. In other embodiments, the information transmission from traditional EEG electrodes 103 to local processing module 108 may be performed wirelessly, and in other embodiments receiving CPU 106 and local processing module 108 may be combined into a single processing module. In one embodiment, the system 102 would initiate monitoring of the subject or patient's EEG data autonomously. In another embodiment, the operator could use display device 104 alone or in conjunction with a digital input device such as a keyboard or computer mouse to enter data and/or select and initiate an exam. A computing system to manage data ingestion, processing, management, and/or annotation may include a local processing module 108 which may use Wi-Fi, Bluetooth or other protocol to access a communication network 110 and interface with a remote processing module 112, other computing system(s) 114, other detector system(s) 116, or database(s) or data repositories 118 to perform the acquisition, ingestion and processing of EEG or other sensor data. In one embodiment of the system, the interpretation of the EEG data could be performed by an algorithm stored on a computer-readable medium either locally on the device's local processing module 108 or on a remote processing module 112. In another embodiment, a qualified human expert may review the raw or processed data and may perform the interpretation via display device 104 or other interface that would enable annotation of features, signatures or other data elements with or without an overall interpretation and summary of the findings. In some embodiments, display device 104 may receive data via receiving CPU 106. In any of the above embodiments, the final interpretation could be returned to the examinee, the operator or another individual requiring the information via the display device, some other method of electronic communication, or by uploading the report to an electronic medical record associated with the subject.

In one embodiment, the traditional EEG detection system 102 includes a plurality of sensors in addition to EEG electrodes 103 including but not limited to: oximetry, sound sensors, light sensors, temperature sensors, gyroscopes, accelerometers and heart-rate sensors. In another embodiment, the device includes a minority of these sensors in addition to the EEG electrodes, or only includes the EEG electrodes with no additional sensors.

FIG. 1B depicts an additional exemplary set of hardware and functional components capable of implementing at least some embodiments described herein. In this example, a head-mounted EEG detector device 126 includes a local processing module 130 that interacts with communication network 110 or plurality of networks such as a Bluetooth or Wi-Fi to communicate with remote processing module 112, electronic medical record 120, database(s) or data repositories 118, other detector system(s) 116, computing or analytics system(s) 114, and display device 132, which includes both a receiving CPU 134 and personal display device 136.

To use this system, the examinee or an examiner, who may be a healthcare provider, technician or other operator would apply the EEG detection device electrode array 128 to the examinee's forehead. In one embodiment, the device would initiate monitoring of the subject or patient's EEG data autonomously. In some embodiments, the system may be capable of monitoring more than one device at a time (e.g., multiple subjects).

In another embodiment, the operator could use the personal display device 136 to enter data and/or select and initiate an exam, where display device 136 may receive data and/or information to display via receiving CPU 134. A computing system to manage data ingestion, processing, management, and/or annotation may include a local processing module 130 which may use Wi-Fi, Bluetooth or other protocol to access a communication network 110 and interface with a remote processing module 112, other computing system(s) 114, other detector system(s) 116, or database(s) or data repositories 118 to perform the acquisition, ingestion and processing of EEG or other sensor data. In one embodiment of the system, the interpretation of the EEG data could be performed by an algorithm stored on a computer-readable medium either locally on the device's local processing module 130 or on a remote processing module 112. In another embodiment, a qualified human expert may review the raw or processed data and may perform the interpretation via personal display device 136 or other interface that would enable annotation of features, signatures or other data elements with or without an overall interpretation and summary of the findings. In any of the above embodiments, the final interpretation could be returned to the examinee, the operator or another individual requiring the information via the display device, some other method of electronic communication, or by uploading the report to an electronic medical record associated with the subject.

In one embodiment, the head-mounted EEG detection system 126 includes a plurality of sensors in addition to EEG electrodes including but not limited to: oximetry, sound sensors, light sensors, temperature sensors, gyroscopes, accelerometers and heart-rate sensors. In another embodiment, the device includes a minority of these sensors in addition to the EEG electrodes, or only includes the EEG electrodes with no additional sensors.

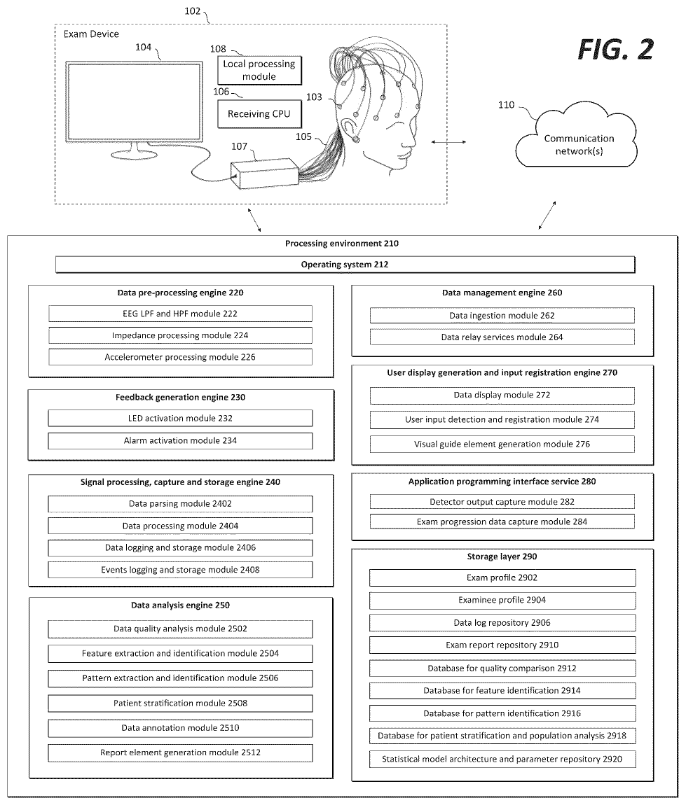

FIG. 2. is a block diagram depicting an exemplary set of functional components, including embodiments of processing environment components capable of implementing various aspects of EEG detection device technology as described herein. In said exemplary set of embodiments, the processing environment 210 may be supported and implemented by either local processing modules 108, or by remote processing module(s) 112 described in FIG. 1 via a communication network 110, or by a combination of local and remote modules. In said embodiments, the processing environment 210 is comprised of an operating system 212, a data pre-processing engine 220, a feedback generation engine 230, a signal processing capture and storage engine 240, a data analysis engine 250, a data management engine 260, a user display generation and input registration engine 270, an application programming interface service 280 and a storage layer 290.

Said operating system 212 establishes the underlying software infrastructure that provides configuration instructions to the hardware components and the local processing module 108 and processes, analyzes, stores and/or otherwise manages data collected by the exam device, wherein the device may be a traditional EEG system 102 or a reduced-montage wireless device 126. Components of said processing environment 210 and said operating system 212 may be implemented across multiple processing modules described in FIG. 1. As an exemplary implementation of the disclosed technology, said data pre-processing engine 220 and said feedback generation engine 230 are implemented with the local processing module enabled by the exam device, whereas said user display generation and input registration engine 270 is implemented on the display devices 104 or 136 described in FIG. 1, and said communications network 110 is used to interface with a remote processing module 112 that implements said signal processing capture and storage engine 240, a remote data repository that implements said storage layer 290, and a remote computing system that implements said data analysis engine 250, said data management engine 260 and said application programming interface service 280. It should be appreciated that various functions may be performed on or off-device or in combination, and some embodiments are not limited to the location of the function performed. Further, it should be appreciated that certain aspects of the system may include a specially-programmed computer, but in some instances, may be a general purpose computer including standard hardware and operating systems, upon which various components described herein may be implemented.

Said data pre-processing engine 220 accesses sensor data that may include but is not limited to EEG, cameras, gyroscopes, accelerometers, oximeters, temperature probes, heart-rate monitors, depth-sensing systems and other sensors or systems. In an exemplary embodiment, an EEG low-pass filter (LPF) and high-pass filter (HPF) module pre-processes data collected by the EEG electrode array. In this embodiment, the data pre-processing engine also includes processing modules for impedance 224 and accelerometer 226 data. In other embodiments, additional processing modules are implemented to process data collected from cameras, gyroscopes, oximeters, temperature probes, heart-rate monitors, depth-sensing systems or other sensors or systems.

Said feedback generation engine 230 controls modules that generate feedback signals that are implemented by the EEG hardware components. In an exemplary embodiment of the disclosed technology, the feedback generation engine 230 enables an LED activation module 232 to control illumination, frequency, duration, color or other parameter for one or a plurality of light-emitting diodes (LEDs) or other light-emitting indicators on the EEG exam device 128 described in FIG. 1B. In said embodiment, the feedback generation engine 230 also enables an alarm activation module 234 to control other types of feedback signals, including but not limited to sound, buzz, haptic feedback or other signal type that may be interpreted by the examinee, examiner or other operator.

Said signal processing, capture and storage engine 240 enables management of pre-processed data that are generated by the data pre-processing engine 220, or, in other embodiments, data that are collected directly from the EEG electrodes or other relevant sensors. Said signal processing, capture and storage engine 240 enables implementation of a data parsing module 2402 and a data processing module 2404, which prepare data for further analysis or storage. The data logging and storage model 2406 and events logging and storage module 2408 interact with the storage layer 290 to capture and store data and events respectively, as determined by the requirements of the exam, or input from the examiner, examinee or other operator.

Said data analysis engine 250 allows real-time or asynchronous assessment of output from the EEG detector device or other sensor. The data quality analysis module 2502 evaluates the data that is being generated by the EEG detector device or other sensors compared to predetermined thresholds or real-time calculated thresholds that indicate sufficient data quality to enable interpretation or other data processing, storage or analysis tasks. In one embodiment, said evaluation is performed with a real-time updated estimator of data quality and an associated confidence estimator.

The feature extraction and identification module 2504 enables raw, pre-processed or processed data to be evaluated for the presence, frequency, characteristics or other parameters of clinical features. One example of a clinical feature that may be detected is an epileptiform spike. The pattern extraction and identification module 2506 enables raw, pre-processed or processed data to be evaluated for the presence, frequency, or characteristics of patterns that are constructed from multiple features, consist of observable changes over time, or represent a statistical association with a parameter or outcome of interest. One example of a pattern that may be used is burst-suppression, a brain state seen in stages of general anesthesia, coma, or hypothermia. The patient stratification module 2508 enables patient-level data to be evaluated in the context of one or a plurality of other patient-level data in order to identify commonalities, differences, sub-group definitions, sub-group characteristics, or other analyses relevant to features, patterns, statistical associations or other analytical conclusions that may be made about a group of individuals.

The data annotation module 2510 creates data annotations that capture the results of data analysis performed by other elements of the data analysis engine 250, aid the system in future data retrieval and/or aid the examinee, examiner, other operator or qualified expert in reading, surveying, interpreting or otherwise interacting with the data. Said data annotation module may communicate with the visual guide element generation module 276 in order to make said annotations available to the examiner, examinee, other operator or qualified expert. The report element generation module converts the analyses performed by other elements of the data analysis engine 250 into elements that are represented in a report describing the results of the exam, in summary format, with detail about specific analytical results or with a combination thereof. Generated report elements may be created as a printed element, electronically generated in a static or dynamic format, or may be modified into any other format by which the results of the exam may be communicated to the examiner, examinee, other operator, qualified expert, family member or other authorized party.

Said data management engine 260 enables data ingestion and relay from multiple sources. In an exemplary embodiment, the data ingestion 262 and relay services 264 modules enable data and analysis results from the signal processing capture and storage engine 240 and data analysis engine 250 to be relayed to the storage layer 290. In said embodiment, the data ingestion module 262 also enables ingestion of EEG or other sensor, laboratory, imaging or other clinical data such that these data may be added to relevant databases in the storage layer 290 and/or accessed by other elements of the processing environment to aid in the performance of analyses, determination of relevant configurations, creation of report elements, or any other action performed by said system.

Said user display generation and input registration engine 270 enables the examinee, examiner, other operator, qualified expert or other authorized party to interact with the system. The data display module 272 generates an interface whereby one or a plurality of said users can view elements including, but not limited to: real-time data streams, results of analyses, warnings or alarms, metrics related to exam quality, metrics related to connectivity, technical information about the system or other information relevant to the performance, management or interpretation of the exam. The user input detection and registration module 274 enables the system to capture a range of inputs from the examinee, examiner, other operator, qualified expert or other authorized party in order to aid the user in making one or a plurality of selections, inputting data, modifying the exam or other functions that require input from the user or aiding the system in the performance of setting exam parameters, setting configuration, data collection, data processing, data analysis, or any other function performed by the system. One or a plurality of said users can provide information via said user input detection and registration module 274 via one or a plurality of interactions including but not limited to: pressing a button, moving a switch, performing a touch-sensitive interaction with a screen, typing information, speaking a command or creating another sound, eye tracking, gesture detection or any other mechanism by which the user communicates information to the system

Said user input detection and registration module 274 also enables one or a plurality of said users to provide information based on prior knowledge, expertise or active problem solving to interpret the data, analysis, report or other artifact generated by the system. In some cases, the information provided by one or a plurality of said users may be to verify the accuracy of the data, analyses or report generated by the system. The visual guide element generation module 276 may utilize data such as information about the exam environment, the examinee, features or signatures within the EEG waveform data or other data available about the patient, exam or data feeds, data collected from the subject's electronic medical record, data stored in the storage layer or other supplementary data. Using these data sources, the visual guide element generation module 276 is able to produce the design, content, appearance and other features of virtual exam guide elements for the purpose of helping the examinee, examiner, other operator, qualified expert or other authorized user to identify relevant features signatures or other observable elements of the data in order to perform an accurate interpretation.

In another embodiment, the interpretation is performed autonomously by an algorithm stored in a computer-readable format or generated de novo for the specified analysis, and the visual elements are used to explain the interpretation that was generated automatically (e.g., through machine learning/AI, or other statistical analysis technique). The visual elements generated by said visual guide element generation module 276 may include but are not limited to text instructions, guiding graphics such as one or more arrows, targets, circles, color-changing elements, progress bars, transparent elements, angles, ‘ghost’ outlines of comparator data signatures, projections of real-time or stored imaging data, or other virtual items. The guiding elements may exist and change according to a predetermined set of instructions, or in response to feedback elements such as navigation through data feeds or new annotations performed by one or a plurality of authorized users, passage of time, acquisition of new exam data, other user input, examinee input/The guiding elements generated by the method may be updated before, during, or after the exam via any of these inputs alone or in combination. The disclosed method may generate or adapt visual or graphical elements in accordance with completion or lack of completion of the exam or a subset thereof.

Storage layer 290 is an integrated data layer that allows high-throughput ingestion, retrieval, and inference of signal data. Encoding is performed by the signal processing and integration engine 240, which in one embodiment of the disclosed method, implements a compression codec that utilizes block-based and pre-entropy-coding inter-channel decorrelation. Said data stored in the storage layer 290 may be used as an input for other functional components of the system. In particular Data may be retrieved from said data analysis engine 250 for inference, interpretation or other subsequent analysis by the signal analysis engine 250.

In one exemplary embodiment, said storage layer 290 maintains a notion of semantically versioned processes that interact with and generate database content (reports, annotations, derived signals/artifacts), a request/response-driven task management workflow for extracting insight from domain experts, a notion of programmable votes/ballots for aggregating expert consensus, and a browser application that facilitates task-oriented machine-learning augmented workflows for biosignal recording labeling, monitoring, and review. Stored data may include, but is not limited to, exam profile data 2902, examinee profile data 2904, a data log repository 2906, an exam report repository 2910, one or a plurality of databases for quality comparison 2912, one or a plurality of databases for feature identification 2914, one or a plurality of databases for pattern identification 2916 and one or a plurality of databases for patient stratification and population analysis 2918, or other stored data types or categories. The statistical model architecture and parameter repository 2920 stores architectures, parameters and any other variables necessary to implement a machine learning or statistical model to identify features, patterns, patient stratification or other clinically relevant read-outs and/or other outputs. Said components of the storage layer may be stored in one or a plurality of distributed machine-readable media connected by a communications network.

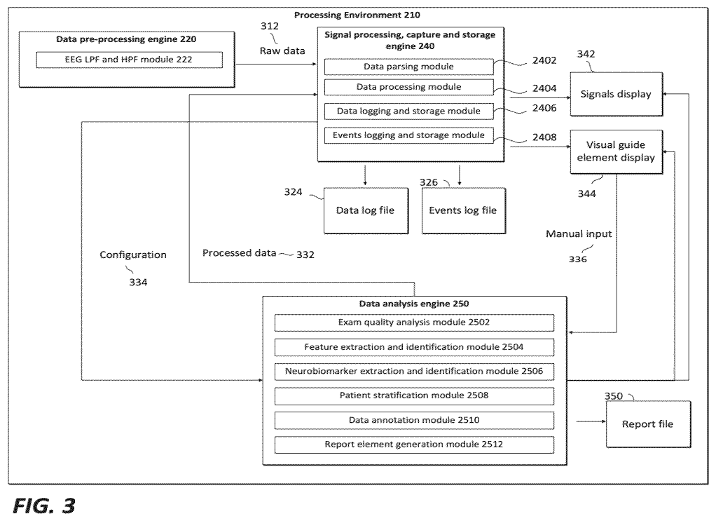

FIG. 3 is a block diagram that depicts an embodiment of the software architecture that enables EEG signals to be communicated between the data-preprocessing engine, signal processing, capture and storage engine and data analysis engine.

In some embodiments, the software architecture described in FIG. 3 is implemented in the processing environment 210, which is supported and implemented by either local processing modules 108 or 130, receiving device CPUs 106 or 134 or by remote processing module(s) 112 described in FIG. 1 via a communication network 110, or by a combination of local and remote modules. In some embodiments, the receiving CPU 106 is integrated with the display device 104 as illustrated in FIG. 1A. In another exemplary embodiment, the receiving CPU 134 is integrated with the display device 134 as illustrated in FIG. 1B. In other embodiments, the receiver CPU and the display device are separate devices and are instead integrated via a communications network 110.

In said exemplary embodiments where the receiver CPU and display devices are integrated into the same device, the Bluetooth Low Energy (BLE) transmitter module 222 communicates data 312 to the receiving device computer processing unit 106 or 134 which implements the signal processing capture and storage engine 240. Although BLE may be used, other communication protocols may be used.

As previously described in FIG. 2, said signal processing capture and storage engine 240 enables implementation of a data parsing module 2402 and a data processing module 2404, which prepare data for further analysis or storage. The data logging and storage module 2406 and events logging and storage module 2408 interact with the storage layer 290 to capture and store data and events respectively, as determined by the requirements of the exam, or input from the examiner, examinee or other operator. Processed data are stored in a data log file 324 and events are stored in an events log file 326. Processed data may also be sent directly to a signal display interface 342 or to a visual guide element display 344. Alternately, processed data shown in path 332 may be sent to the data analysis engine 250. In one exemplary embodiment of the disclosed technology, the data analysis engine is implemented on a remote computing system 114 described in FIG. 1. In another exemplary embodiment, the data analysis engine 250 is implemented on the receiver and display device. In yet another embodiment, some or all of the modules comprising said signal processing capture and storage engine 240 and said data analysis engine 250 are implemented on the local processing unit 130 of the head-mounted detector device. As described in FIG. 2, said data analysis engine 250 allows real-time or asynchronous assessment of output from the EEG detector device or other sensor, including data quality analysis 2502, feature extraction and identification 2504, pattern extraction and identification 2506, patient stratification 2508, data annotation 2510 and/or report element generation 2512. One or a plurality of analysis results generated by said data analysis modules working alone or in combination may be deposited in the form of a summary or detailed report in a report file 350 which is then stored in the storage layer 290. Generated report elements may be simultaneously or asynchronously created as a printed element, electronically generated in a static, dynamic, or interactive format, or may be modified into any other format by which the results of the exam may be communicated to the examiner, examinee, other operator, qualified expert, family member or other authorized party.

One or a plurality of analysis results generated by said data analysis modules working alone or in combination may alternately or simultaneously be communicated to the signals display 342 or to the visual guide element display 344. In an exemplary embodiment of the disclosed technology, the visual guide elements are displayed in conjunction with the underlying signal such that the visual guide elements highlight, annotate, or otherwise identify portions of the underlying data that contribute to the summary results of the analysis. Said visual guide elements may also indicate the confidence, strength of association or other indication of relativity with regards to the resulting assessment. One or a plurality of analysis results generated by said data analysis modules working alone or in combination may alternately or simultaneously be processed to supply configuration instructions 334 to said signal processing, capture and storage engine 240, or subsequently as configuration instructions 310 to the Bluetooth Low Energy transmitter 222 to update the instructions, function, methods, algorithms or other settings implemented by the local processing modules 108 or 130 described in FIG. 1.

In the aforementioned exemplary embodiment where the receiver CPU and display devices are integrated into the same device, the device is configured with sufficient local data storage for at least 14 days continuous recording within the storage layer 290. Alternatively, the receiving and display device is configured to offload portions of the data using communications network(s) 110 to a cloud storage solution to reduce the amount of data that is stored locally. In said embodiment where data is offloaded to a cloud storage solution, the duration of data that may be stored by the system is limited only by the availability of cloud storage. For said local data storage, said cloud data storage, or other form of data storage, clinical exam data are stored using a common standardized format and encryption protocol such that the same data ingestion and interpretation methods may be used to post-process data stored by multiple instances or embodiments of the disclosed system. The receiver and display devices 132 or 102 as disclosed herein are also capable of initiating and maintaining real-time data streams of signals that are collected from the acquisition device which are then communicated to a cloud-based computing and storage platform, either directly over an encrypted connection 110 if external network access is available, or via protected intra-network endpoints that securely relay necessary outgoing/incoming connections. Said device is enabled with encryption algorithms to reconstitute data communicated by the acquisition device and/or to encrypt data that is subsequently communicated to other components of the system, such as a cloud-based remote computing module or data storage solution 118. Said encryption algorithms are of sufficient quality to prevent any unauthorized third party from intercepting and reconstituting transmitted data into a usable form that would in any way contain personally-identifiable health data or any data on the status or function of any aforementioned components of some embodiments. In the circumstance that a third party attempted to intercept said encrypted data transmission the system is capable of recognizing such interference, halting data transmission and notifying the examinee, examiner, other operator or system administrator of said interference. Said device is enabled with Wi-Fi, a cellular SIM card, or any other communication device to communicate with said cloud storage solution and for other communication with central data repositories 118 or computing systems 114 as described in FIG. 1. In said embodiment, the receiver and display devices 132 or 102 also contain a camera that may be used in addition to or instead of the integrated light meter on the acquisition device to detect ambient light or other light-based signals. Examples of hardware that would enable at least one embodiment include but are not limited to a tablet computing device or a smartphone device.

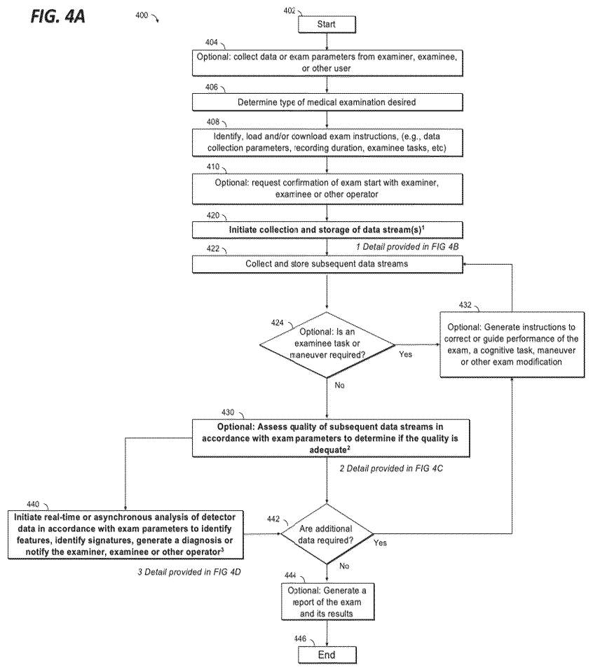

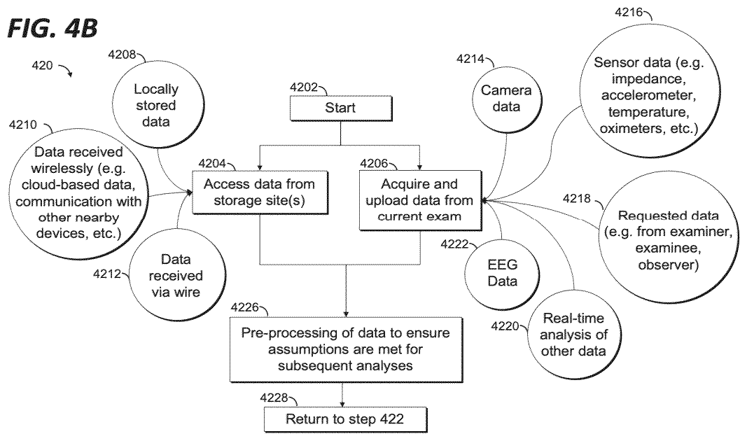

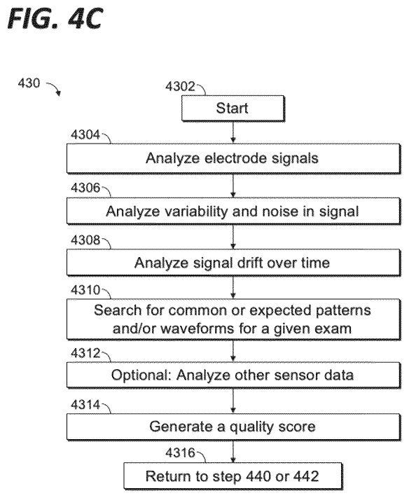

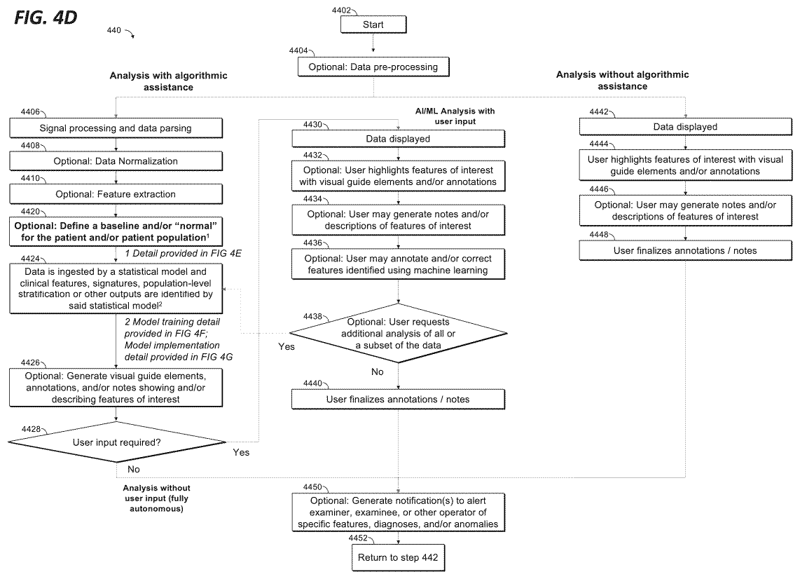

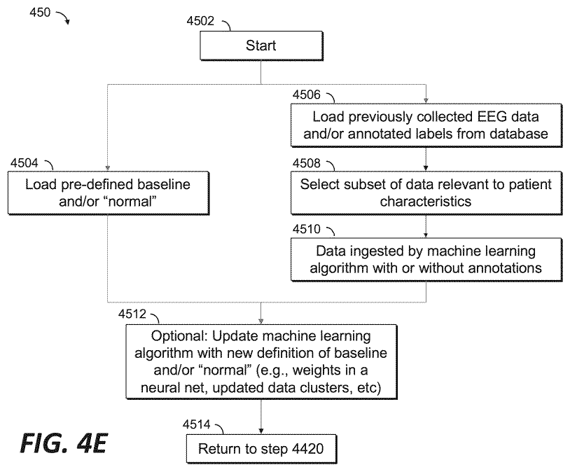

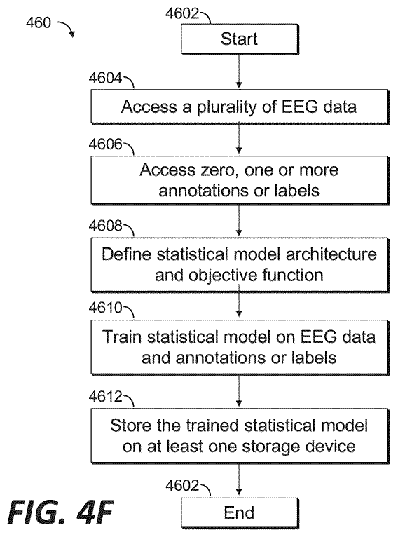

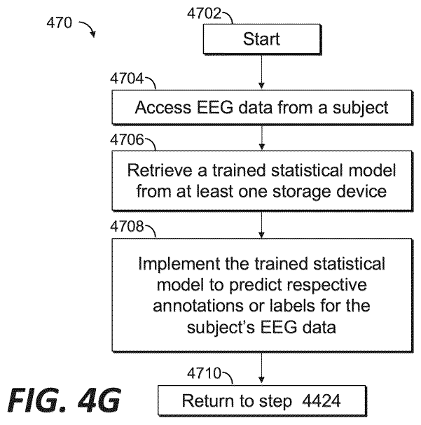

FIG. 4A is a flow chart illustrating an embodiment of a method implemented by the system to accomplish the general goal of collecting appropriate data streams, analyzing said data and optionally generating a report describing the results of said analysis.

Method 400 may be performed locally on an electronic device such as local processing module 108 depicted in FIG. 1A, online via a cloud system, via remote processing module 112, via computing system(s) 114, or via some combination thereof. The method begins at 402. At 404, data or exam parameters may be optionally collected from an examiner, examinee, or other user. Examples of data or exam parameters to be collected may include but are not limited to the exam duration or relevant patient or clinical information. It should be appreciated that these examples are a non-exhaustive list of possible data and/or parameters and the system maintains flexibility such that the collection of a plurality of data and/or parameters not explicitly listed herein is possible and intended as a feature of the system. At 406 the system determines the type of medical examination desired through user input, pre-loaded instructions, or other means. At 408, exam instructions are identified, loaded, and/or downloaded if necessary. Exam instructions may include but are not limited to data collection parameters, recording duration(s), and/or required examinee tasks. At 410 the system optionally requests confirmation to start an exam with input from the examiner, examinee, or other operator, after which the exam commences with the system initiating collection and storage of data stream(s) at 420. Additional details for step 420 are provided in FIG. 4B, which may include collection of data from one or both of storage sites and/or data from the current exam such as EEG data, camera data, other sensor data, requested data, or real-time analysis of other data. Any subsequent data streams are collected and stored at step 422, which may continue to implement some or part of step 420 throughout the exam and for the duration of the method. In one embodiment, both steps 420 and steps 422 are implemented in part or in whole by signal processing, capture, and storage engine 240 as depicted in FIG. 2.

If a specific exam maneuver is required by the examiner, examinee, or other user at step 424, then at step 434 the system may optionally generate instructions to correct or guide performance of said maneuver. In some embodiments, maneuvers may include but are not limited to cognitive tasks, such as picture naming, visualization, speaking, remembering a number, word, or shape, or tracing a figure by hand. Maneuvers may include physical movements, repositioning, or other maneuvers that may benefit the exam as well. In some embodiments, instructions may include written word or text, while in other embodiments said instructions may include visual guide elements shown on a display device. Other embodiments may include some combination thereof. If a maneuver is required, data is collected at step 422, following optional instruction generation at step 432. If multiple maneuvers are required, step 424, optional step 434, and step 422 may be repeated until no additional maneuvers are required at step 424. If no additional maneuver is required, then the method proceeds to step 430 at which the quality of subsequent data streams is assessed in accordance with exam parameters to determine if the quality of said data is adequate for the desired outcome. Additional detail for step 430 is provided in FIG. 4C. Assessing the quality of data is important to ensure high-quality output and results for the exam. A quality score is generated in step 430 that may be used to determine if additional data is required at step 442. For example, if data quality of one or more data streams is deemed inadequate to conclude an exam or result(s) with confidence, additional data may be requested, at which point the system may optionally generate instructions in an attempt to improve exam performance and/or quality at step 432, followed by repetition of steps 422 and 424. In some embodiments of the system, a quality score may be generated at step 430, but it may be necessary to collect additional information before the system can determine if additional data is required at step 442. In such an embodiment, the next step performed may be step 440. In other embodiments, the exam instructions, examiner, or other user may intentionally dictate the desire to process data of poor quality and proceed to step 440 regardless of the quality score. If the system determines that the data is of adequate quality and/or the quality score has met a specific threshold, it may also proceed to step 440. At step 440, data collected throughout the exam is analyzed in accordance with exam parameters to identify features, identify signatures, generate a diagnosis, and/or notify the examiner, examinee, or other operator of findings. In some embodiments, the analysis in step 440 may be performed in real-time as examination data is collected, while in other embodiments the analysis in step 440 may be performed asynchronously from the time of exam data collection. If analysis is performed in an asynchronous manner, step 440 may be skipped until all data collection has been completed. Additional detail describing step 440 is provided in FIG. 4D, which details embodiments in which analysis is performed in a fully autonomous manner by a machine learning algorithm or artificial intelligence, embodiments in which analysis is performed by a human reader, and embodiments that are a combination thereof.

In one embodiment, some or all of the analysis occurs online via a cloud system. In another embodiment, the analysis may be performed by remote processing module 112 or computer system(s) 114. In another embodiment some or all of the analysis occurs on a local processing unit (e.g., local processing unit 108 or 130 as detailed in FIG. 1A or FIG. 1B, respectively). The results of said analysis performed in step 440 may be sent to receiving CPU 106 or 134 and display device 104 or 136. Alternately, the results of analyses may be used to activate or control relevant feedback modules 230, in such a way that a feedback signal is generated including but not limited to a dynamic change in the LED to indicate detection of a feature or pattern. One example of a feature is an epileptiform spike. An example of a pattern is burst suppression. A local processing unit 108 or 130 may be programmed to generate specific feedback signals based on the type, frequency, duration or other parameter of the feature or pattern detected, for example by illuminating one or more LED's to indicate seizure activity, by generating a buzz to indicate a pending seizure, by illuminating an LED of a specific color or playing a specific sound to indicate that the examinee is a specified sleep stage such as rapid eye movement (REM) sleep, or providing biofeedback or stimuli to the subject at particular stages of sleep. In another embodiment, a signal such as an illuminated LED, sound, or buzz is generated to indicate poor contact for one or more electrodes, or to indicate a corrective action required to improve signal quality.

Following analysis at step 440, the system, exam instructions, examiner, other user(s), analysis result, or data quality may dictate that additional data is required at step 442. If additional data is required, the system proceeds to step 432. Otherwise, if no additional data is required, the system proceeds to step 444 where a report of the exam and its results may be optionally generated. In some embodiments, report generation occurs via report element generation module 2512 described in FIG. 2, which may utilize the results of said analyses performed in step 440 to generate elements that may be included, recorded and logged in a summary or detailed report describing the results of the exam. At 446, the method ends.

FIG. 4B is a flow chart showing detail of step 420 from FIG. 4A, in which necessary data inputs are collected and integrated.

Method 420 may be performed locally on an electronic device such as local processing module 108 depicted in FIG. 1A, online via a cloud system, via remote processing module 112, via computing system(s) 114, or via some combination thereof. The method begins at 4202. At step 4204, data is accessed from appropriate storage sites, including locally stored data 4208, data received wirelessly (for example, cloud-based data) 4210, and/or data received by wire 4212, including but not limited to EEG or other sensor data from previous exams that have been rated for data quality, identification of relevant features, appropriate sensor placement, or adequacy of maneuver performance. In another embodiment, data accessed at step 4204 may include pre-trained weights for a neural network or other data employed by machine learning algorithms known to one skilled in the art. At step 4206, the system acquires and uploads data from the current exam, including data from camera sensors 4214, sensor data 4216 (e.g., including but not limited to impedance, gyroscope, accelerometer, temperature, or oximeter data), requested data provided via manual input 4218 (e.g., inputs from the examiner/user, examinee, or other user or observer), real-time analysis of other data 4220, EEG data 4222. In various embodiments, some, none, or all of the aforementioned data may be acquired and uploaded from the current exam. At step 4226 these data are pre-processed to ensure that basic assumptions are met for subsequent analysis. For example, in one embodiment the system may use incoming data in a calibration step to determine whether or not the incoming sensor data satisfies the system's assumptions pertaining to acquisition of data. At step 4228, the method ends and the system returns to step 422 described in FIG. 4A.

FIG. 4C is a flow chart showing detail of step 430 from FIG. 4A, in which exam data quality is assessed. Method 430 may be performed locally on an electronic device such as local processing module 108 depicted in FIG. 1A, online via a cloud system, via remote processing module 112, via computing system(s) 114, or via some combination thereof. In some embodiments, method 430 may be performed leveraging data analysis quality module 2502 as depicted in FIG. 2. The method begins at 4302. At step 4304, electrode signals are analyzed. Analyses of electrode signals may include but are not limited to measuring impedance, voltage, signal offset (indicating poor electrode to skin contact), measurements of kurtosis or skewness (EEG is considered to have a gaussian distribution), measuring signal fidelity across electrodes, measurements of line noise, measurements of electromyography and electrooculography artifacts, measuring signal correlation across electrodes, other features, or some combination thereof. In some embodiments, the system may extract features and/or patterns that are relevant for quality assessment and compare the extracted features and/or patterns to data stored in a database or repository and/or that have been assessed by individuals skilled at EEG interpretation. In said embodiment, the system may integrate global data quality assessment metrics and feature and/or pattern comparisons against stored data to create an integrated score of the current signal quality using a predefined threshold and/or threshold(s) derived from cumulative assessment across signals in a database or repository.

In some embodiments, analyses of electrode signals may include one or more machine learning algorithms known to one skilled in the art. In one embodiment a neural network trained to recognize common EEG artifacts may be employed. A neural network may recognize very low-frequency oscillations consistent with sweat artifact, small spike-like discharges associated with rapid eye movements, glossokinetic potential from tongue movement, spiky waveforms associated with talking, chewing artifact, movement artifact, pulse artifact, electrode “pop” artifact, or a variety of other EEG artifacts known to one skilled in the art. In some embodiments, a machine-learning algorithm could be designed to function as an integrated classifier and grade EEG signals as “adequate” or “inadequate” based on features learned from a training database composed of EEG signals previously assessed by individuals skilled in EEG interpretation. Said methods could similarly be applied to data streams from one or a plurality of sensors, such as in step 4312, including but not limited to accelerometers, oximeters, temperature probes, gyroscopes or other sensors. In another embodiment, said methods are used to create mixed models that generate outputs based on a plurality of sensor inputs or clinical metadata such as lab values, demographics, radiology reports, pathology reports, clinical assessments or other clinical data.

In one embodiment, recognition of these artifacts may be done in conjunction with an EEG safety read examination, particularly when artifacts can simulate seizure-like activity. Recognition of false seizure-like activity is particularly important when testing the safety of a new therapeutic, where false safety alarms may result in unnecessarily halting a clinical trial. At step 4306, variability and noise in the signal are analyzed. Variability and noise may be present if an electrode connection is loose, one or more electrodes is faulty, one or more electrodes is making poor surface contact with the skin, or for a plurality of other reasons. Analyzing variability and noise helps ensure the fidelity of the signal. Other common calculations known to one skilled in the art may be performed at 4306, such as calculating a signal to noise ratio. At step 4308, signal drift over time is analyzed. Signal drift may occur if the gel used at the interface of the electrode and the skin is not stable, or if there is poor skin contact. At step 4310 the system may search for common or expected patterns and/or waveforms for a given exam. In one embodiment, the system may search for waveforms expected in conjunction with a maneuver, specific patterns based on the given exam, or other commonly studied waveforms such as delta, theta, alpha, sigma, and beta waves. At step 4312 the system may optionally analyze other sensor data including but not limited to accelerometer, oximeter, temperature probe, gyroscope or other sensor data. In one embodiment, gyroscope and/or accelerometer data may be used in conjunction with EEG data to increase confidence when identifying movement artifact. In some embodiments, a moisture sensor may be used to increase confidence in sweat artifact. At step 4314, the system uses the previous analysis performed in previous steps to generate a quality score, metric, or decision. In some embodiments, data may be compared to predetermined threshold or real-time calculated threshold that indicate sufficient data quality to enable interpretation or other data processing, storage, or analysis tasks. In some embodiments, said evaluation is performed with a real-time updated estimator of data quality and an associated confidence estimator. In another embodiment, data pertaining to the quality of exam data or to the analysis of features, patterns, signatures or patient stratification interpretation may be communicated via a communication network to experts skilled in the art, such as neurologists, for manual quality interpretation or for verification of the accuracy of automated interpretation or results. In said embodiment, the automated quality interpretation and results could be alternatively used to calibrate or score the accuracy of expert interpreters, or to train other individuals to perform quality interpretation. At 4316 the method ends and the system returns to step 440 or step 442 described in FIG. 4A. In one case, the system may determine the quality of data is too low to perform analysis and return to step 442, while in another case the system may find the quality of data adequate for further analysis and return to step 440. In another case, exam parameters may dictate that analysis at step 440 is to be performed regardless of the quality outcome, and in yet another case exam parameters may dictate otherwise.

FIG. 4D is a flowchart showing detail of step 440 from FIG. 4A, in which data streams are analyze with or without assistance from machine learning statistical models or with optional user input in conjunction with machine learning statistical models.

Method 440 may be performed locally on an electronic device such as local processing module 108 depicted in FIG. 1A, online via a cloud system, via remote processing module 112, via computing system(s) 114, or via some combination thereof. The method begins at 4402. At 4404, the system optionally performs a data pre-processing operation using data pre-processing engine 220. In said data pre-processing operation, methods commonly known to one skilled in the art may be employed. Said methods may include but are not limited to dimensionality reduction, feature encoding, feature sampling, feature aggregation, identification of inconsistent values, removal of corrupted data, and/or identification of missing values. In an alternate embodiment, data preprocessing is performed in step 430 of method 400 and further preprocessing is not required in method 440. The system may then proceed with Analysis with algorithmic assistance by proceeding to 4406 or Analysis without algorithmic assistance by proceeding to 4442. In an exemplary embodiment, user input may be collected in step 404 or 406 of method 400 or elsewhere to determine whether method 430 should proceed with algorithmic assistance via 4406 or without algorithmic assistance via 4442.

In step 4406, the system performs signal processing and data parsing methods commonly known to one skilled in the art. In an exemplary embodiment, step 4406 includes parsing data via one or more data transforms. An example of such a data transform includes a Fourier transform in which signal is decomposed into frequency components for further analysis. Said decomposition and analysis may be performed by the signal processing, capture and storage engine 240 described as a component of the system's processing environment 210. In said exemplary embodiment or in another exemplary embodiment of step 4406, the system may parse data into discretized components and/or convert one data file type to another. The system may perform pre-processing techniques to filter, sort, denoise, clean or organize the data for further analysis and display. At 4408, the system may perform data normalization. Data normalization is commonly employed when assessing multiple data inputs with varying scales and/or absolute values. In one such embodiment, the system may normalize various frequency bands, pulse oximeter data, accelerometer data, and/or other data. At 4410, the system optionally performs feature extraction. In some embodiments, ML algorithms could be incorporated for various functions such as for example, spike burden, population-level interpretation at step 4222 and 4224, or other aspects of functionality. In an exemplary embodiment, the system may calculate the derivative of a given signal, reduce the dimensionality of a feature-rich dataset to decrease future processing requirements, and/or employ other common feature extraction techniques known to one skilled in the art. At 4420, the system optionally defines a baseline and/or “normal” comparator for further analysis of the index patient or patient population. Details of sub-method 4420 is provided in FIG. 4E described below.

At 4424, data are ingested for analysis by one or multiple machine learning or statistical models implemented by the data analysis engine 250 described as a component of the system's processing environment 210. In one embodiment, said one or multiple machine learning or statistical models are trained using method 460 from FIG. 4F, described below. In one embodiment said one or multiple machine learning or statistical models are implemented using method 470 from FIG. 4G, described below.

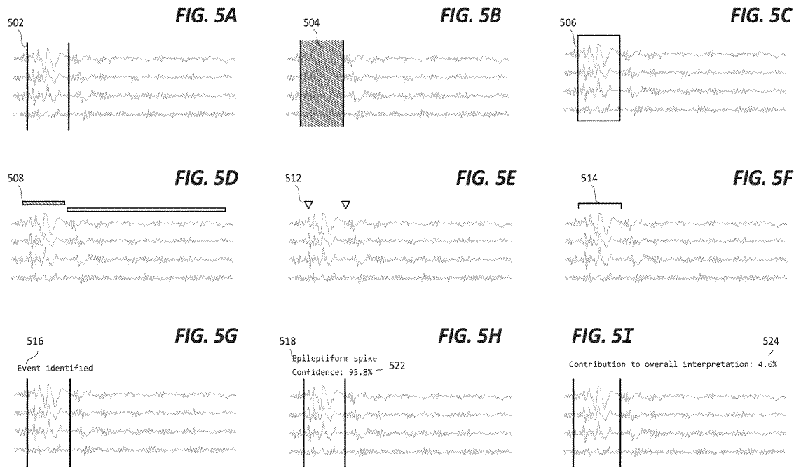

Once the system has identified one or more clinical features, the system may optionally generate visual guide elements, annotations, and/or notes showing and/or describing said features at step 4426. Several example embodiments of visual guide elements and/or annotations are shown in FIGS. 5A-5I. In other embodiments, explicit written notes and/or other forms of annotation may be used to alert a user and/or reader to a feature. It should be appreciated that FIGS. 5A-5I and the aforementioned elements should not be considered an exhaustive list of possible annotations and/or guide elements that may be employed by the system claimed herein. At step 4428 the system determines if user input is required. In an exemplary embodiment, the system is capable of triaging specific data to automated algorithmic interpretation or human expert interpretation based on the confidence level output from the initial run of model(s). For example, using this approach, if the algorithm delivers an interpretation with high confidence, then a fully automated workflow may be implemented and in such an embodiment step 4450 would be subsequently performed following step 4428. If the confidence of the algorithmic result is of significantly lower confidence, then the data may be delivered to a human expert clinician for interpretation and in such an embodiment step 4430 would be subsequently performed following step 4428. In said exemplary embodiment, the confidence threshold may be established via user input collected in step 404, 406 or elsewhere. In another embodiment, the confidence threshold may be determined algorithmically based on population-level frequencies, statistics or other estimation. In said exemplary embodiment, virtual guide elements are generated to indicate the relevant data points that support the algorithm's conclusion as well as additional data points that contribute to the confidence estimation. In one embodiment, the system may notify multiple human experts that an interpretation is required, followed by implementation of a request for user input by which one of said group of multiple human experts confirms his or her availability and willingness to perform said human interpretation. In another embodiment, more than one human expert may provide interpretation of the data. In said embodiment if the interpretations of the multiple human experts are in agreement, then a consensus interpretation will be available to the system for subsequent processing and report generation. If the interpretations of the human experts differ significantly, then yet another human expert may be notified to provide an additional interpretation to generate a consensus interpretation.

Each human expert may additionally be provided with the interpretations of the other human experts in order to consider alternate interpretations. In another embodiment, the system may enable digital communication mechanisms such as peer-to-peer chat, audio voice call technology, video call technology or asynchronous written communication to enable the multiple human experts to provide the rationale for their interpretation such that the group of multiple experts may discuss the data and determine an adjudicated final consensus interpretation. In other embodiments, an automated consensus measure may be performed, and in some implementations, this measure may evaluate a consensus threshold (e.g., similar to the confidence threshold). In some embodiments, contacting of human experts may be based on the threshold and/or other measures (e.g., cost).

In another embodiment of the disclosed system and method, human experts are provided with digital tools to create and share virtual guide elements to identify components of the underlying data that contributed to their interpretation and communicate these findings to the one or more additional human experts. Said virtual guide elements may appear as static or dynamic and may be integrated with said audio, video or written communication systems. In each of the aforementioned embodiments in which human interpretation and/or user input is required, the system would implement step 4438 following implementation of step 4430 and determination of whether user input is required. In any embodiment in which human interpretation is not required, the system would proceed to step 4450 (i.e. Analysis without user input (fully autonomous).

If the system has determined that user input is required and proceeds to step 4430, then data is subsequently displayed to one or more users. Data may include but is not limited to EEG data, results of previous analyses, other sensor data, other user interpretations and/or annotations, and/or guide elements and/or annotations generated in previous steps by the system claimed herein. Data may be displayed via display device 104 and/or personal display device 136 as shown in FIGS. 1A and 1B. At step 4432, one or more users may optionally highlight features of interest in the data using visual guide elements and/or annotations. In one exemplary embodiment, said user(s) may employ guide elements similar to those displayed in FIGS. 5A-5I. In a similar embodiment and/or another exemplary embodiment, said user(s) may use other methods of annotation including but not limited to freeform graphical drawings, audio recordings, video recordings and/or digital tagging. Following optional feature highlighting and/or annotation, said user(s) may further annotate and/or describe findings through notes and/or written descriptions at step 4434. In some embodiments user(s) may use written word, while in the same or other embodiments, user(s) may employ audio recordings or other forms of annotation. At step 4436, user(s) may annotate and/or correct features identified by one or more machine learning algorithms and/or statistical models. In some embodiments, this information is used in step 4438 to help direct subsequent analyses. At step 4438, one or more users may optionally request additional analyses of all or a subset of data. In some embodiments, once this option is requested, the system may further request user input to identify specific desired analyses, identify data subsets of interest, clarify information related to user interpretation discrepancies between a previously outcome or feature generated by said system and/or other users, and/or convey other information to one or more user(s) such as diagnostic information relevant to understanding a discrepancy between one or more user(s) and said system or user feedback such as time until analysis completion. If one or more users requests additional analysis, step 4424 is subsequently performed, followed by steps 4426, and 4428, at which point the system determines whether further user input is required. Step 4428 may be performed in a similar manner to one or more aforementioned embodiments. In some embodiments, system 4428 may use information collected from one or more users in step 4438 to determine if further user input is required. If one or more users does not request further analysis at step 4438, the system proceeds to step 4440 at which point the system prompts one or more users to finalize his or her annotations and/or notes. In some embodiments, all users are required to finalize annotations and/or notes before the system proceeds to step 4450. In other embodiments, some, one, or no users are required to finalize annotations and/or notes before proceeding to step 4450. In some embodiments, step 4440 may serve as a mechanism to prompt step 4450.

Following step 4404, if the system determines Analysis without algorithmic assistance is desired, then at step 4442 data is subsequently displayed to one or more users. Optionally, at or before step 4442, a data presentation step may be performed that presents data for functions such as unit conversions, friendly names, and data visualization related transformations (e.g. gain, band filtering, log scale). Data may be displayed via display device 104 and/or personal display device 136 as shown in FIGS. 1A and 1B. Data may include but is not limited to EEG data, results of previous analyses, other sensor data, and/or other user interpretations and/or annotations. As previously described herein, the system may include one or multiple users. In exemplary embodiments in which multiple users are included, several previously described embodiments are relevant for steps 4442 through 4448. For example, in some embodiments multiple human experts may be notified, requested to interpret one or more data elements, and/or confirm availability and/or willingness to perform data interpretation. In the same or other embodiments, multiple interpretations may be used to provide a consensus interpretation available for subsequent processing and report generation. If the interpretations of users differ significantly, then yet another user may be notified to provide an additional interpretation to generate a consensus interpretation. Each user may additionally be provided with the interpretations of other users in order to consider alternate interpretations. In another embodiment, the system may enable digital communication mechanisms such as peer-to-peer chat, audio voice call technology, video call technology or asynchronous written communication to enable the multiple users to provide the rationale for their interpretation such that the group of multiple experts may discuss the data and determine an adjudicated final consensus interpretation. In another embodiment of the disclosed system and method, users are provided with digital tools to create and share virtual guide elements to identify components of the underlying data that contributed to their interpretation and communicate these findings to the one or more additional users. Said virtual guide elements may appear as static or dynamic and may be integrated with said audio, video or written communication systems.

At step 4444, one or more users may optionally highlight features of interest in the data using visual guide elements and/or annotations. In one exemplary embodiment, said user(s) may employ guide elements similar to those displayed in FIGS. 5A-5I. In a similar embodiment and/or another exemplary embodiment, said user(s) may use other methods of annotation including but not limited to freeform graphical drawings, audio recordings, and/or digital tagging. Following optional feature highlighting and/or annotation, said user(s) may further annotate and/or describe findings through notes and/or written descriptions at step 4446. In some embodiments user(s) may use written word, while in the same or other embodiments, user(s) may employ audio recordings or other forms of annotation. At step 4448, the system may prompt one or more users to finalize his or her annotations and/or notes. In some embodiments, all users are required to finalize annotations and/or notes before the system proceeds to step 4450. In other embodiments, some, one, or no users are required to finalize annotations and/or notes before proceeding to step 4450. In some embodiments, step 4448 may serve as a mechanism to prompt step 4450.

At step 4450 the system optionally generates notification(s) to alert an examiner, examinee, and/or other operator of specific features, diagnoses, and/or anomalies. In one exemplary embodiment, the system may identify an epileptiform spike and subsequently generate an alert. This finding may be included in report generation step 444 in method 400; however, an alert such as an audible noise, visual alert, or otherwise may be employed to more promptly alert an examiner, examinee, or other operator to said finding in order to facilitate appropriate action such as providing a therapeutic intervention or ceasement of a causative intervention. At step 4452, the system returns to step 442.

FIG. 4E is a flowchart of showing detail of step 4420 from FIG. 4D, in which a baseline and/or “normal” comparator is established for the purposes of evaluating the current data streams against said baseline and/or “normal” comparator.

Method 450 starts at 4502. In one embodiment a computer-readable storage medium stores a pre-defined baseline and/or “normal” database. Said database may be downloaded or accessed in step 4504 for further analysis. Alternately, in step 4506, the system may access previously collected EEG data and/or annotated labels from a computer-readable storage medium. In step 4508, the system may select a subset of the data relevant to characteristics of the patient data currently being analyzed. Subsequently in step 4510, the data are ingested by the machine learning or statistical algorithm with or without said annotations. In said exemplary embodiment, user input may be collected in step 404 or 406 of method 400 or elsewhere. In another embodiment, the baseline and/or “normal” comparator and/or comparator based on previously collected EEG is a statistical summary such as a high-dimensional vector, matrix or array representation of underlying comparator data.

In step 4512, the system may optionally update the machine learning or statistical algorithm or model with a new definition of baseline and/or “normal”. In said exemplary embodiment, said operation involves updating parameter weights in a neural net, updating data clusters or performing analyses to determine characteristics of the test population versus the comparator population established in step 4504 or 4508. In step 4514, the system returns to step 4420.

FIG. 4F is a flow chart showing a method for training a statistical model used in step 4424 from FIG. 4D. Method 460 starts at 4602. At step 4604, a plurality of EEG data are retrieved from at least one computer-readable storage medium. Said EEG data may be from a single subject, a group of subjects or a plurality of groups of subjects. In one embodiment, to train a model on epileptiform spike detection, EEG data may be obtained from a group of subjects known to demonstrate epileptiform EEG activity. In another embodiment, to train a model to differentiate between Alzheimer's disease and frontotemporal dementia, a plurality of EEG data may be obtained from one group carrying an Alzheimer's disease diagnosis and another group carrying a frontotemporal dementia diagnosis. In some embodiments, a plurality of EEG data may also be obtained from healthy normal controls. At step 4606, zero one or more annotations or labels are retrieved from at least one computer-readable storage medium. Annotations may be performed by one skilled in the art of EEG interpretation, for example a trained clinical epileptologist. Some annotations may be comprised of interpretations from multiple experts, along with a consensus interpretation. In another embodiment, EEG segments with morphologic similarity to expert-labeled segments may be labeled in an automated fashion and added to the training dataset. Alternately annotations may be comprised of related clinical data or endpoints such as video assessment of a clinical seizure, cognitive scoring results, behavioral scoring results, lab data, imaging data, clinical response to treatment, patient survival or other relevant data. If zero annotations or labels are retrieved and used in training, then the model would be considered an unsupervised statistical or machine learning model. At step 4608, a model architecture and an objective function are defined. It should be appreciated that the statistical or machine learning model could be constructed using a range of methods, could be trained with multiple methods to compare performance and select a preferred architecture, or could implement multiple model architectures simultaneously in series, in parallel or via some other combination. Suitable model architectures are chosen from a range of architectures known to one skilled in the art. In one embodiment, the machine learning model is a deep neural net consisting of convolutional, pooling and fully-connected layers. In other embodiments, the machine learning or statistical model architecture is one of the group comprised of the following: a generalized linear model, a random forest, a support vector machine, and/or a gradient boosted tree. Methods known to one skilled in the art may be implemented to perform dropout, normalization and regularization. In one embodiment, an objective function is selected to assess the performance of the model and optimize performance over the course of training.

In step 4610, the statistical model is trained on the plurality of EEG data and the one or more annotations or labels. EEG data may be segmented into smaller segments (e.g., 1 second) during training. For example, models may be trained to interpret features such as those that a create burst suppression severity index, model changes in spike burden over time, stratify patients based on electrophysiologic subgroup, or other clinically-relevant output. Further, predictive models may be trained to generate an alert output when a change in the burst suppression severity index (for example) is likely to increase or when a subject is likely to enter a certain sleep stage, predict progression of cognitive decline over time, predict correlations with protein-based biomarkers, predict correlations with imaging findings, predict response to one or more therapies, predict adverse effects from one or more therapies or other clinically-relevant predictions or prognoses.

Models may be trained using a plurality of EEG data from step 4604 in a supervised or unsupervised manner depending on the zero, one or more annotations or labels that are retrieved from step 4606. Machine learning model(s) may be prespecified or selected by one or more users for a specific analysis. In one embodiment, said features may be identified autonomously by an algorithm stored in a computer-readable format or identified de novo for the specified analysis. In an exemplary embodiment, said machine learning models and/or statistical models may identify epileptiform spikes indicative of seizure-like activity, generalized slowing, atypical triphasic waves, and/or multifocal sharp waves indicative of neurotoxicity, 60 Hz artifact, interictal epileptiform spikes, burst suppression, small sharp spikes and/or a plurality of other EEG features indicative of normal and/or pathologic disease states known to one skilled in the art of EEG interpretation. Some number of said EEG features may be referred to by synonyms known to one skilled in the art, for example small sharp spikes may alternately be referred to as benign sporadic sleep spikes (BSSS) or benign epileptiform transient of sleep (BETS). Said machine learning models and/or statistical models may also identify EEG features or signatures that may not be recognized by an expert skilled in the art of EEG interpretation, but may be identified to be predictive of, correlate with or are otherwise related to prognosis, therapeutic response, adverse events or other clinically-relevant observations.

FIG. 4G is a flow chart showing a method for implementing a statistical model in step 4424 from FIG. 4D in which data is ingested by a statistical model and clinical features, signatures, population-level stratification or other outputs are identified by said statistical model. Method 470 starts at 4702. At step 4604, EEG data is accessed from a subject. At step 4706, a statistical or machine learning model trained using method 450 or some other method is retrieved from at least one computer-readable storage device. At step 4708, the statistical or machine learning model is implemented to predict respective annotations or labels from the subject's EEG data accessed in 4704. At step 4710, the method returns to step 4424 in method 440 described in FIG. 4D.

The statistical or machine learning model trained using method 460 or some other method may be employed in a clinical trial or general clinical setting according to seven exemplary embodiments: 1) Identify patients with Alzheimer's disease and/or discriminate between patients with Alzheimer's disease and other neurodegenerative diseases, 2) identify or predict patient subgroups with regard to neurophysiology, likelihood of cognitive decline or other clinically-relevant grouping, 3) screen patients prior to enrollment for inclusion or exclusion criteria, 4) identify or predict correlations with other clinically-relevant biomarkers such as protein-based biomarkers or imaging features, or clinical assessments such as cognitive scoring systems, 5) identify indication for therapeutic intervention, 6) identify signals associated with clinical benefit or predict clinical benefit following exposure to one or more therapies, and/or 7) identify signals associated with adverse events or predict adverse events following exposure to one or more therapies.

In the first embodiment of said group of exemplary embodiments, wherein the system and method is implemented for the purpose of identifying patients with Alzheimer's disease and/or discriminating between patients with Alzheimer's disease and other neurodegenerative diseases, said statistical or machine learning model trained in method 460 and implemented in method 470 is configured to identify EEG features, signatures or patient stratifications that are associated with relevant disease state. For example, in one embodiment said statistical machine learning model is trained on sleep EEG such that differences in sleep architecture (e.g., the duration of time spent in relevant REM, N1 or N2 sleep stages, or frequency or pattern of changes between stages) may be used to distinguish between Alzheimer's disease, frontotemporal dementia or other related neurodegenerative disease.

In the second embodiment of said group of exemplary embodiments, wherein the system and method is implemented for the purpose of identifying or predicting patient subgroups, said statistical or machine learning model trained in method 460 and implemented in method 470 is configured to identify EEG signatures that are statistically correlated with or predictive of neurophysiologic sub-groups, likelihood of cognitive decline or other clinically-relevant grouping. Said subgroups may include, but are not limited to, patients with more rapid clinical or cognitive decline, patients with slower clinical or cognitive decline, patients more or less likely to exhibit or develop clinical seizures, patients more or less likely to exhibit or develop behavioral disturbances and/or patients more or less likely to develop sleep disturbances.

In the third embodiment of said group of exemplary embodiments, wherein the system and method is implemented for the purpose of screening patients for inclusion or exclusion criteria, said statistical or machine learning model trained in method 460 and implemented in method 470 is configured to identify clinical features, signatures or patient stratifications that are associated with relevant disease features (e.g., clinical or sub-clinical epilepsy). In one embodiment, a disease state may be pre-specified by trial investigators as criteria either for inclusion or exclusion in an investigational study (e.g., to test an investigational drug for the treatment of Alzheimer's patients with known epilepsy, or to exclude Alzheimer's patients with epilepsy who may be at higher risk of adverse effects from a therapy aimed at treating a different condition). Alternatively, the machine learning or statistical model(s) may be trained or otherwise configured to identify patients most likely to benefit from an investigational therapeutic. For example, if a mutation in a given gene locus is known to increase risk of rapid cognitive decline by inducing either gain-of-function or loss-of-function, then the machine learning algorithm may be trained to segment these populations in order to determine likelihood of response to a given therapy aimed at inhibiting or enhancing the activity of the protein encoded by said gene locus. Implementation of one or more machine learning algorithms of this nature in a clinical trial setting would support development of a companion diagnostic that would ultimately identify patients likely to benefit from a given therapeutic intervention as an indication for treatment, as discussed in other embodiments in this section.

In the fourth embodiment of said group of exemplary embodiments, wherein the system and method is implemented for the purpose of identifying or predicting correlations with other clinically-relevant biomarkers, said statistical or machine learning model trained in method 460 and implemented in method 470 is configured to identify EEG signatures that are statistically correlated with or predictive of other biomarkers such as protein-based biomarkers (for example, amyloid-beta detected in cerebrospinal fluid), imaging features (for example, grey or white matter volume determined by MRI), functional imaging features (for example, fMRI activity during a cognitive task), pathologic features (for example amyloid-beta or tau deposition noted on examination of brain tissue), and/or clinical cognitive scoring systems (for example, the standardized Montreal Cognitive Assessment). Said predictive EEG biomarkers may be utilized, for example, as surrogate biomarkers to avoid the need for frequent invasive or logistically-burdensome exams, predictive of future findings at an earlier timepoint, or to improve predictive accuracy for an outcome of interest in combination with one or more of said biomarkers.

In the fifth embodiment of said group of exemplary embodiments, wherein the system and method is implemented for the purpose of identifying indications for therapy, said statistical or machine learning model trained in method 460 and implemented in method 470 is configured to identify neurophysiologic features that may be used as an indication for initiation, switching or discontinuing therapy and/or may be used in the creation of an overall plan of care. Said indication may be predicated on inclusion or exclusion in a given sub-group, or may be determined on an individual basis. Said indication may be for the initiation of therapy in the general sense, may specify a given therapeutic class defined by mechanism of action or other grouping, may specify a specific therapeutic agent, or may specify a combination therapy defined either by single or multiple therapeutic classes or single or multiple therapeutic agents.

In the sixth embodiment of said group of exemplary embodiments, wherein the system and method is implemented for the purpose of identifying efficacy of a therapeutic intervention, said statistical or machine learning model trained in method 460 and implemented in method 470 is configured to identify clinical features, signatures, patient stratification or other signals that are associated with a response or lack of response to the intervention. Said determination may take into account differences between an individual's baseline and treated states, differences between treated and untreated individuals, baseline control data, normal-comparative database or other comparator. Said therapeutic response may be performed in the context of a clinical trial or in the context of broader clinical practice.

In the seventh embodiment of said group of exemplary embodiments, wherein the system and method is implemented for the purpose of identifying adverse events, said statistical or machine learning model trained in method 460 and implemented in method 470 is configured to identify clinical features, signatures or other signals that are associated with an adverse effect of a therapeutic intervention, an adverse effect of the patient's underlying disease condition, an adverse effect of some other medication or exposure, or any other adverse effect. Said adverse events could represent a risk to the patient or a reportable event identified in a clinical trial's protocol or safety monitoring plan, identified in a healthcare institutional safety program, or identified in a national or international safety program. In an exemplary embodiment, one or more machine learning algorithms and/or statistical models may be configured to identify generalized periodic discharge, generalized rhythmic delta activity, slowing, and/or generalized spike-and-waves, any of which may indicate drug-induced neurotoxicity.

FIGS. 5A-I are examples of virtual guide elements that are used to identify features or signatures for the purpose of identifying diagnostically-relevant signals or patterns.

The system and method described herein is capable of generating a wide range of virtual guide elements. Said elements may be generated as the output of a statistical model trained on a large volume of EEG data in order to identify segments of data of likely diagnostic importance, or to directly annotate said segment with a diagnostic interpretation. Alternatively, an expert human interpreter may generate a virtual guide element to mark his or her own expert interpretation or annotation such that another operator, another expert, the patient or the original expert may subsequently view the virtual guide element. Virtual guide elements may appear in a wide range of form-factors, including but not limited to the exemplary embodiments illustrated in FIGS. 5A-I. In FIG. 5A, the virtual guide element 502 employs boundaries to highlight the data span of interest. In FIG. 5B, the virtual guide element 504 employs a transparent colored or textured element to highlight the data span of interest. In FIG. 5C, the virtual guide element 506 employs an enclosed object to highlight the data span of interest. In FIG. 5D, the virtual guide element 508 employs a superimposed band to highlight the data span of interest. In FIG. 5D, the virtual guide element 508 employs a superimposed band to highlight the data span of interest. In FIG. 5E, the virtual guide element 512 employs a superimposed pointers to highlight the data span of interest. In FIG. 5F, the virtual guide element 508 employs a superimposed bracket to highlight the data span of interest. In FIG. 5G, the virtual guide element 516 employs text in combination with geometric virtual guide elements to annotate the data span of interest. In FIG. 5H, the virtual guide element 518 employs text to annotate the type of event identified, along with a text-based indicator of confidence 522 in combination with geometric virtual guide elements to annotate the data span of interest. In FIG. 5I, the virtual guide element 524 employs text to identify the relevance of the highlighted span for the overall interpretation of the data collected, along with an indicator of the relevant weight of the contribution.

In all of said exemplary embodiments of virtual guide elements, said elements may be colored, textured, sized, or otherwise highlighted or enhanced to make them visually appealing, easy to locate, or to communicate information such as event category. In another exemplary embodiment of the technology, said virtual guide elements are not static, but dynamically respond to the collection of data, addition of new annotations or other user or automated inputs.

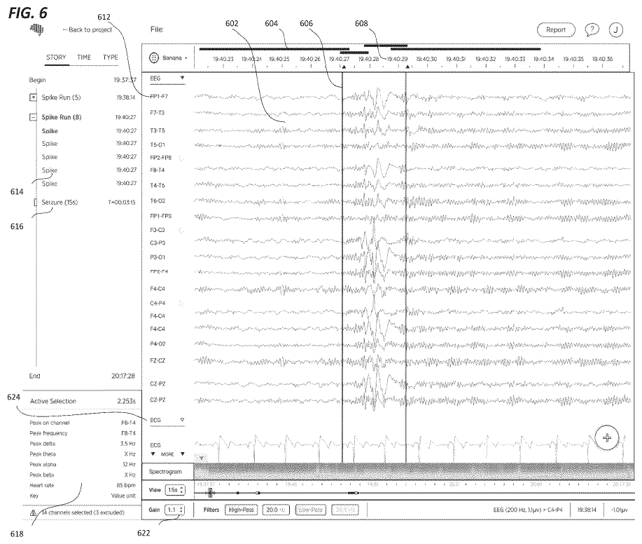

FIG. 6 is an exemplary embodiment of the display interface that would enable an expert to interrogate the data captured by the detection device with the assistance of virtual guide elements and the ability to annotate or confirm diagnostic interpretations.

In said exemplary embodiment, an expert is presented with a virtual dashboard that enables visualization and navigation of the raw recorded data 602, optionally segmented or organized by the relevant electrode 612 or alternate data stream, such as an electrocardiogram lead 624. The interface displays visual guide elements, such as automated annotations 604 that are created by statistical models to identify segments of raw data that have a given probability of identifying diagnostically-relevant information. Other virtual tools 606 enable the operator to manually annotate segments of interest to add expert interpretation or to confirm automatically annotated interpretations. The data is viewed with relevant time stamps 608 enabling ease of navigation. In said exemplary embodiment, the interface also displays a consolidated log of events or spans of diagnostic relevance 614, which may include automated annotations, manual annotations or both. Said element in the interface may also display a diagnostic interpretation such as “seizure” 616, which may similarly be generated by automated interpretation, manual interpretation or a combination of automated and manual inputs. An additional interface element 618 summarizes overall statistics from the relevant collected data or any defined subset thereof. In said embodiment, the interface also includes additional tools to define key signal processing parameters 622 including but not limited to: gain, high-pass filter value, low-pass filter value or time window to analyze.

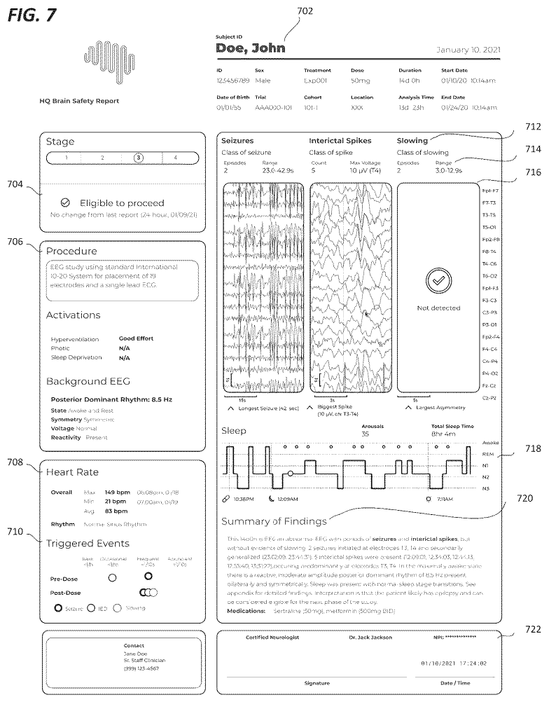

FIG. 7 is an exemplary embodiment of a report that describes the results of the system's analysis with or without user input as generated by the method described in FIG. 4A. In one embodiment an identifier 702 indicates the patient or trial subject described in the report. In other embodiments, the report may describe results from multiple patients defined by the overall study population or a sub-group thereof. In an exemplary embodiment of the report element 704 describes an overall summary of the conclusion of the report such as an indicator that the patient meets inclusion and exclusion criteria to be enrolled in a given clinical trial. In an exemplary embodiment of the technology, the report includes an element 706 that describes the parameters of the study performed including study requirements, procedures performed and summary metrics of baseline data collected during the study. In an exemplary embodiment of the technology, element 708 provides summary statistics of ancillary sensors or data collected via standard clinical or biometric devices, such as heart rate summary statistics, electrocardiography summary statistics, height, weight, blood pressure, accelerometer summary statistics, gyroscope summary statistics, fluid balance summary statistics, temperature summary statistics, respiration rate summary statistics, pulse oximetry summary statistics or other vital signs or clinical metrics.

In an exemplary embodiment of the report, element 710 represents a graphical summary of electroencephalographic events that have occurred during the recording period. In said exemplary body, the graphical summary identifies clinically-relevant EEG events that have occurred prior to administration of a therapeutic agent and after administration of said agent such that the reader may interpret the summary effect of said therapeutic agent. Exemplary events include seizure activity, interepileptic discharges, power spectral band slowing, generalized slowing, or other electroencephalographic features or signatures that are interpretable to a specialist or non-specialist clinician. In an exemplary embodiment of the report, element 712 provides additional detail on specific EEG signatures of interest. Element 714 describes quantitative metrics associated with the analysis, such as pre-defined spectral band ranges. Element 716 provides a graphical snapshot with primary EEG data that were used to construct the interpretation. In one embodiment element 716 presents selected examples of EEG data representative of the interpretation. In another embodiment, element 716 presents all examples of EEG data that were used to construct the interpretation.

In one embodiment of the report, element 718 demonstrates a summary time-based EEG activity profile for the relevant patient, subject or patient group. In one embodiment, said EEG activity profile demonstrates the time relationships of interpretations presented in elements 710, 712 and 716. In another embodiment, the EEG activity profile is a sleep profile. In yet another embodiment, the EEG activity profile is an interactive timeline that the user can manipulate to highlight the time points associated with specific interpretations in relation to other timepoints over the course of the study period.

In said exemplary embodiment of the report, element 720 is a text summary of the findings. Said text may be automatically generated by a statistical machine learning algorithm. Said text may alternately be written or dictated by a clinician skilled in the art of EEG interpretation consistent with the interpretation delivered in the report. Element 722 provides a signature or alternate affirmation of the authenticity of the report. In an exemplary embodiment, said affirmation is a signature of a clinician who has reviewed and approved the interpretation automatically generated by a statistical or machine learning model. In another embodiment, said affirmation is a signature of a clinician who has generated the report based on said clinician's expert skill in the art of EEG interpretation and interpretation of associated data.

In some embodiments, the report is stored in a computer-readable memory in a static format such as a .pdf document or equivalent digital document. In said embodiment, the report may be converted to a physical document using standard printing technology. In another embodiment, the report may be stored in a computer-readable memory with a dynamic format in which elements may be programmed to be responsive to user input. Said input may be performed using a standard mouse-based graphical user interface where user input may include hovering a cursor, clicking, dragging, typing hotkey commands, typing text, or manipulating controls such as buttons, scroll bars, text fields, zoom tools, selection tools, or tab navigation tools. In another embodiment, said input may alternately be performed using touchscreen-based technology, where user input may include tapping, swiping, pinching, scrolling or other hand gestures.

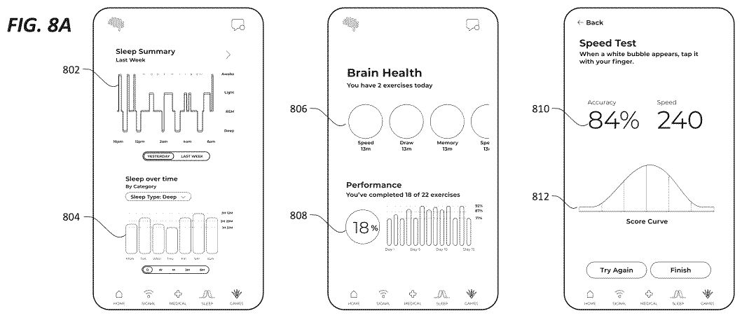

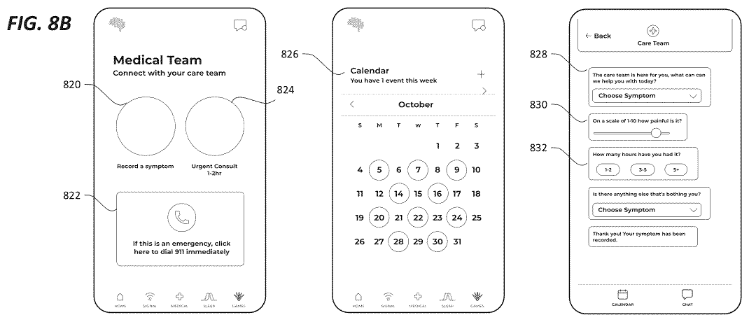

FIGS. 8A-B are exemplary summary illustrations of visual elements that are presented to the patient or examinee using the disclosed system and method. The disclosed system is capable of presenting multiple information types to the patient, examinee or examiner via a display system that may resemble a smartphone, tablet, mobile computer, desktop computer or other display device. In one embodiment, the system generates summaries of sleep staging 802 and quantified sleep metrics over time 804. In another embodiment, the system is programmed to host specific cognitive tests 806 that may include widely validated clinical scoring systems. Said cognitive tests may variously include tests to assess reaction speed, cognitive and mechanical skills associated with drawing or reproducing images, memory or recall tests, numerical or mathematical tests, pattern recognition tests, language interpretation tests, or any other relevant tests. After completion of one or more tests, further visual elements may be generated to display specific performance metrics 810, performance contextualized against past performance or broader patient populations, or other relevant summative metrics relevant to tests that have been performed. In one embodiment, the system may also generate an interface 820 that the patient, a family member or other individual may use to record relevant medical information such as a new or recurring symptom. Additional interface elements may be displayed that enable a user, family member or other individual to schedule an urgent consultation with a medical professional 824 or to contact emergency services 822. Other interface elements generated by the system enable a patient, family member or other individual to see a calendar or schedule 826 that may be used to display relevant medical events or tasks such as study visits for a clinical trial, cognitive testing events, or routine visits with a medical professional. If a patient or study subject is experiencing a symptom, the system may also be configured to provide visual elements to aid the individual in recording a symptom, which may be accessed, for example, through visual element 820. Symptom recording tools may include drop-down menus 828, sliders 830, pre-set scales 832, customizable elements, text fields or other elements that aid the patient, user or other individual to record symptom information in a structured or unstructured format.

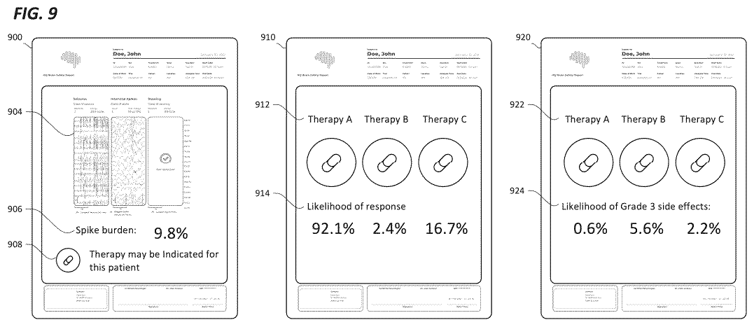

FIG. 9 Is an exemplary summary illustration of report elements that are presented to the examiner or physician using the disclosed system and method. 900 is an exemplary illustration of a report focused on presenting information about whether a given therapy or class of therapies may be indicated for a given patient based on the results of EEG events 904, or quantitative EEG biomarkers such as epileptiform spike burden 906, alone or in combination. Epileptiform spike burden is an exemplary neurobiomarker that reflects event frequency of epileptiform discharges detected by EEG either as an independent metric or as a function of time, percent of study period or other quantitative benchmark. In other embodiments, reported neurobiomarkers may include epileptiform spike density, frequency, morphology or any other features that are identified to be relevant to pathophysiology, treatment, treatment side-effects cognitive function, disease progression or other clinical features of Alzheimer's disease. In exemplary illustration 900, the system is shown generating visual elements that reflect an epileptiform spike burden 906, and a companion diagnostic read-out 908 suggesting that the patient may be a candidate for therapy. The system may additionally or alternately be configured to generate prediction metrics, including likelihood of progression of cognitive, behavioral or other relevant clinical symptoms. In exemplary illustration 910, the system is shown generating visual elements that reflect multiple therapies 912 for which the patient or study subject may be a clinical candidate. Additionally the system generates visual elements reflecting consolidated estimates of the likelihood of clinical benefit 914 from each therapy. In exemplary illustration 920, the system is shown generating similar visual elements that reflect multiple therapies 922, paired with visual elements reflecting consolidated estimates of the likelihood of significant adverse side effects 924 from each therapy. Said efficacy or safety likelihood estimates may reflect a combination of EEG-based neurobiomarkers such as epileptiform spike burden, clinical or cognitive testing scores, imaging, protein-based biomarkers, demographic data or any other clinical data to which the system has access. Estimates may be static or may vary in real time as new data are collected from a given patient or as underlying statistical models are updated based on other patients, autonomous updates or technical improvements. In one embodiment, the system is configured to perform population-level assessments such as stratification, grouping or clustering, which may include identification of sub-groups that are more likely to benefit from an intervention, might be at higher risk of an adverse event, or any other clinically-relevant outcome. An example of a sub-group is Alzheimer's patients that demonstrate subclinical epileptiform discharges, but do not demonstrate clinical seizures. In the event that the system provides a read-out on a patient's candidacy for a given therapeutic or therapeutic class based on likelihood of clinical benefit, risk of adverse events or other clinical evaluation, the system would serve the function of a companion diagnostic.

The system may additionally or alternately be configured to Identify correlations between EEG-based neurobiomarkers and other established biomarkers such as MRI imaging-based biomarkers, protein biomarkers such as amyloid-beta in cerebrospinal fluid (CSF) or other biomarker. In another embodiment, the system is configured to assess one or more of EEG-based neurobiomarkers, cognitive scoring tests, demographic data, protein-based biomarkers, imaging or any other relevant clinical data in order to evaluate for trial eligibility, including assessment of inclusion or exclusion criteria. In another embodiment, the system may be configured and implemented to perform monitoring for adverse safety events in a clinical trial setting (e.g., verifying that initial doses of a drug did not cause seizure-like activity) or in a routine clinical setting. In another embodiment the system may be configured to monitor for efficacy of a given intervention, for example reduction in epileptiform spike burden.

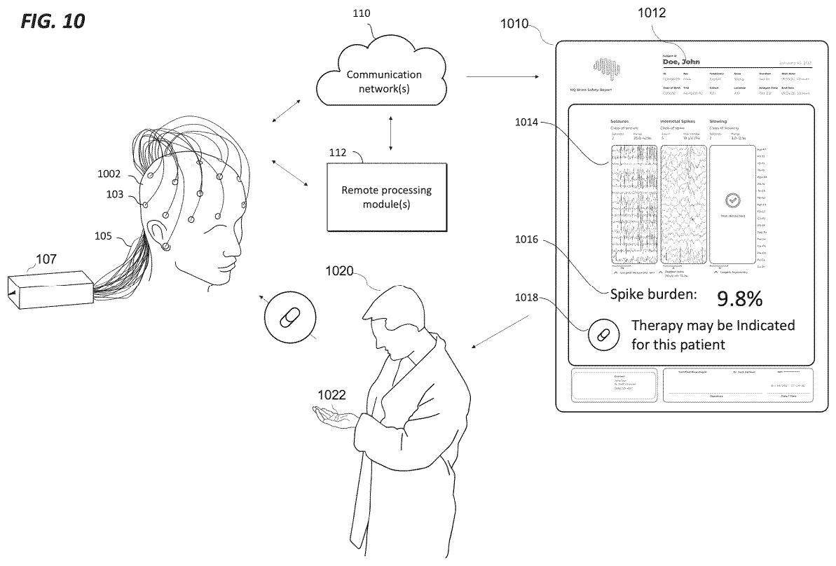

FIG. 10 is an exemplary summary illustration of the communication workflow of the disclosed system and method. In said exemplary communication workflow, a subject 1002 is monitored with an exemplary embodiment of the disclosed system and method, including components of an EEG detection device 103, 105 and 107 described in FIG. 1. Subject 1002 may be a research subject in the setting of a clinical trial or may be a patient receiving routine medical care. Subsequently the communications network 110 remote processing module 112 and remainder of the system and method described in FIGS. 1-4 are implemented to generate a report 1010. In the exemplary illustration, report 1010 includes relevant identifying information 1012 for subject 1002 along with detailed read-outs from the EEG recording period 1014, consolidated metrics describing epileptiform spike burden 1016 and a companion diagnostic read-out that therapy is indicated for subject 1002. A clinician 1020 subsequently provides an intervention 1022 to the patient or research subject 1002. In an exemplary embodiment, said intervention 1022 may be an investigational therapeutic drug given in the context of a clinical trial. In another exemplary embodiment of the disclosed system, a display device receives an interpretation rendered by the analysis system implemented in the processing environment described in FIG. 2. In said exemplary embodiment, the display device renders the patient's EEG data to the clinician 1020 with a virtual guide elements.

In various embodiments, the system may perform other functions using EEG and/or other types of signals. For instance, the system may perform compliance functions such as user verification, security processes, activity logging features and other compliance features. Further, the system may be used during different use cases in general clinical practice or in the setting of clinical trials. For instance, the system may be used to perform safety reads, may provide companion diagnostics, used to measure clinical features of interest, used for measuring enrollment criteria, or other clinical use.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In some embodiments, a machine learning model, artificial intelligence model or other type of statistical model may be used to determine environmental parameters to be used to process one or more EEG or other signal types. In one implementation, a model may be trained based on EEG signals. Certain outcomes, diagnoses, or other outputs may be also used to train the model and permit the model to predict outcomes based on the input parameters. The model may be part of a computer system used to provide indications to one or more users. Other implementations and systems can be used that take EEG signals to appropriately predict one or more outcomes.

In this respect, it should be appreciated that one implementation of the embodiments comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments. The computer-readable storage medium can be transportable such that the program stored thereon can be loaded onto any computer resource to implement the aspects discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects.

Various aspects may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having the same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting.

The terminology utilized herein is intended to describe specific embodiments only and is in no way intended to be limiting of the invention. The term “and/or”, as used herein, includes any and all combinations of one or more of the associated listed items. As used herein, the singular forms “a,” “an,” and “the” are intended to include the plural forms as well as the singular forms unless the context clearly indicates otherwise. As used herein, the terms “comprises” and/or “comprising” are intended to specify the presence of features, elements, or steps, but do not preclude the presence or addition of one or more additional features, elements, and/or steps thereof. The term “device” as used herein should be interpreted as a broad term defined as a thing adapted for a particular purpose. This includes but is not limited to medical examination tools such as EEG electrodes, EKG electrodes, oximetry probes, temperature probes, but also other things a user may utilize in performing an exam such as a user's own hands, ears, nose, or eyes. The terms “electroencephalography electrode” or “EEG electrode” should be understood to mean an electrode capable of detecting electrical activity, including depolarization and repolarization of neuronal tissue spatially arranged in the central nervous system, including various regions of the brain. Such electrical activity detected by an EEG electrode is generally thought by those skilled in the art to represent the summed electrical activity of a plurality of neurons acting synchronously or asynchronously individually, in groups, or in networks that are spatially arranged in the area detected by the electrode.

Unless defined otherwise, all terms used herein have the same meaning as commonly understood by one having ordinary skill in the art to which the invention relates. Furthermore, it will be understood that terms should be interpreted as having a meaning that is consistent with their meaning in the context of the relevant art and the present disclosure and will not be interpreted in an overly formal or idealized sense unless expressly defined herein.

To avoid confusion, this description will refrain from repeating every possible combination of features, elements, and/or steps. However, it will be understood that any and all of the listed features, elements and/or steps each has individual benefit and can also be used in conjunction with one or more, or in some cases all, of the other disclosed items, particularly in the claims. Thus, the specification and claims should be read with the understanding that any of these combinations are entirely within the scope of the invention and the claims.

What is claimed is:

1. An electroencephalography (EEG) processing system for training one or more statistical models to analyze neurophysiology associated with Alzheimer's disease, the system comprising:
an EEG detector device comprising an array of sensors;
one or more processors;
a computer-readable storage media coupled to the one or more processors; and
an analysis pipeline implemented by the one or more processor configured to ingest and process electrical signals from the EEG detector device
wherein said one or more computer-readable storage media are further configured to store processor-executable instructions that, when executed by the at least one computer hardware processor, cause the at least one computer hardware processor to perform:
retrieving a trained statistical model from at least one storage device, wherein the trained statistical model is trained on a plurality of annotated EEG data, wherein the plurality of EEG training data includes at least one annotation describing an entity of interest selected from a group comprising:
specific dementia diagnosis,
cognitive scores,
behavioral scores,
rate of cognitive decline,
survival time,
drug response,
patient level phenotype,
genetic mutations,
protein biomarkers,
imaging biomarkers,
likelihood of adverse reaction,
inclusion or exclusion criteria for a clinical trial;
processing, using the first trained statistical model, EEG data from a subject to generate relevant output labels for said entity of interest; and
storing the predicted entity of interest on the at least one storage device.

2. The EEG processing system of claim 1, wherein said one or more computer-readable storage media are further configured to store processor-executable instructions that, when executed by the at least one computer hardware processor, cause the at least one computer hardware processor to perform:
retrieving a first trained statistical model from at least one storage device, wherein the trained statistical model is trained on a plurality of annotated EEG data, wherein the plurality of EEG training data includes at least one annotation describing EEG features or signatures for at least one segment of EEG waveforms;
processing, using the first trained statistical model, EEG data from a subject to generate relevant output labels for EEG features or signatures;
extracting values for one or more features or signatures from the EEG data annotated by the first trained statistical model;
retrieving a second trained statistical model from the at least one storage device, wherein the second trained statistical model is trained on extracted values for the one or more features from the plurality of annotated EEG data;
processing, using the second trained statistical model, the values for the one or more features extracted from the annotated EEG data to predict an entity of interest from said group of entities of claim 1; and
storing the predicted entity of interest on the at least one storage device.

3. The EEG processing system of claim 2, wherein said EEG features or signatures are one or more from the group comprising:
epileptiform discharges;
seizures;
power spectral frequencies;
small sharp spikes; and
sleep spindles.

4. The EEG processing system of claim 2, wherein said values for one or more features or signatures are one or more from the group comprising:
number of epileptiform discharges;
rate of epileptiform discharges;
topographic distribution of epileptiform discharges;
amplitude of epileptiform discharges;
number of seizures;
duration of seizures;
changes in power spectral frequency distributions;
number of small sharp spikes;
rate of small sharp spikes;
topographic distribution of small sharp spikes;
amplitude of small sharp spikes;
number of sleep spindles;
rate of sleep spindles; and
topographic distribution of sleep spindles.

5. The EEG processing system of claim 2, wherein said prediction of said entity of interest is provided to a clinician as a companion diagnostic as an indication for a therapy for Alzheimer's disease.

6. The EEG processing system of claim 2, wherein at least one of the first or second trained statistical model is a convolutional neural net.

7. The EEG processing system of claim 2, wherein at least one of the first or second trained statistical models include a generalized linear model, a random forest, a support vector machine, and/or a gradient boosted tree.

8. An electroencephalography (EEG) processing system for training one or more statistical models to analyze neurophysiology associated with Alzheimer's disease, the system comprising:
an EEG detector device comprising an array of sensors;
one or more processors;
a computer-readable storage media coupled to the one or more processors; and
an analysis pipeline implemented by the one or more processor configured to ingest and process electrical signals from the EEG detector device,
wherein said one or more computer-readable storage media are further configured to store processor-executable instructions that, when executed by the at least one computer hardware processor, cause the at least one computer hardware processor to perform:
accessing a plurality of training annotated EEG recordings associated with a group of patients in a randomized controlled clinical trial or an observational study, wherein each of the plurality of training annotated EEG recordings is associated with diagnosis data for a respective patient, wherein each of the plurality of training EEG recordings includes at least one annotation describing an EEG feature signature or characteristic category for a portion of the recording, wherein the plurality of training annotated EEG recordings includes:
a first plurality of annotated EEG recordings associated with a first group of patients with Alzheimer's disease, and
a second plurality of annotated EEG recordings associated with a second group of patients belonging to a control group without Alzheimer's disease or with a specific alternate diagnosis;
training one or more statistical models based on said plurality of training EEG data and said plurality of annotations;
storing the one or more trained models on at least one storage device; and
processing, using the one or more trained models, EEG data to predict the diagnosis of a new individual or new group of patients not previously used to train said one or more statistical models.

9. The electroencephalography (EEG) processing system of claim 8, wherein said plurality of annotated training EEG recordings includes:
a first plurality of annotated EEG recordings associated with a first group of patients with Alzheimer's disease with accelerated cognitive decline, and
a second plurality of annotated EEG recordings associated with a second group of patients belonging to a control group with Alzheimer's disease but without accelerated cognitive decline;
and said computer-readable storage media are further configured to store processor-executable instructions that, when executed by the at least one computer hardware processor, cause the at least one computer hardware processor to process, using the one or more trained models, EEG data to predict accelerated cognitive decline in a new individual or new group of patients not previously used to train said one or more statistical models.

10. The electroencephalography (EEG) processing system of claim 8, wherein said plurality of annotated training EEG recordings includes:
a first plurality of annotated EEG recordings associated with a first group of patients with Alzheimer's disease that meet inclusion and exclusion criteria for a clinical trial, and
a second plurality of annotated EEG recordings associated with a second group of patients belonging to a control group with Alzheimer's disease but do not meet inclusion and exclusion criteria for a clinical trial;
and said computer-readable storage media are further configured to store processor-executable instructions that, when executed by the at least one computer hardware processor, cause the at least one computer hardware processor to process, using the one or more trained models, EEG data to predict whether a new individual or new group of patients not previously used to train said model meet inclusion and exclusion criteria for a clinical trial.

11. The electroencephalography (EEG) processing system of claim 8, wherein said plurality of annotated training EEG recordings includes:
a first plurality of annotated EEG recordings associated with a first group of patients with Alzheimer's disease that are treated with a therapy, and
a second plurality of annotated EEG recordings associated with a second group of patients belonging to a control group with Alzheimer's disease but are not treated with the same therapy;
and said computer-readable storage media are further configured to store processor-executable instructions that, when executed by the at least one computer hardware processor, cause the at least one computer hardware processor to process, using the one or more trained models, EEG data to predict whether a new individual or new group of patients not previously used to train said model is treated with said therapy.

12. The electroencephalography (EEG) processing system of claim 8, wherein said plurality of annotated training EEG recordings includes:
a first plurality of annotated EEG recordings associated with a first group of patients with Alzheimer's disease that are treated with a therapy, and
a second plurality of annotated EEG recordings associated with a second group of patients belonging to a control group with Alzheimer's disease but are not treated with a different therapy;
and said computer-readable storage media are further configured to store processor-executable instructions that, when executed by the at least one computer hardware processor, cause the at least one computer hardware processor to process, using the one or more trained models, EEG data to predict the clinical response of a new individual or new group of patients not previously used to train said model to either said therapy used to treat the first group of patients or said therapy used to treat the second group of patients.

13. The electroencephalography (EEG) processing system of claim 8, wherein said plurality of annotated training EEG recordings includes:
a first plurality of annotated EEG recordings associated with a first group of patients with Alzheimer's disease that are treated with a therapy, and
a second plurality of annotated EEG recordings associated with a second group of patients belonging to a control group with Alzheimer's disease but are not treated with a different therapy;
and said computer-readable storage media are further configured to store processor-executable instructions that, when executed by the at least one computer hardware processor, cause the at least one computer hardware processor to process, using the one or more trained models, EEG data to predict the likelihood of adverse events of a new individual or new group of patients not previously used to train said model to either said therapy used to treat the first group of patients or said therapy used to treat the second group of patients.

14. A method comprising acts of:
receiving electroencephalography (EEG) signals associated with a subject;
processing, by a statistical model, the EEG signals associated with the subject; and
performing a determination, by the statistical model, of an annotation of the EEG signals associated with the subject relevant to neurophysiology associated with Alzheimer's disease.

15. The method according to claim 14, wherein the annotation includes one or more of a group comprising:
specific dementia diagnosis,
cognitive scores,
behavioral scores,
rate of cognitive decline,
survival time,
drug response,
patient level phenotype,
genetic mutations,
protein biomarkers,
imaging biomarkers,
likelihood of adverse reaction, and
inclusion or exclusion criteria for a clinical trial.

16. The method according to claim 14, further comprising an act of identifying, responsive to the determination, whether the subject has Alzheimer's disease.

17. The method according to claim 14, further comprising an act of identifying, responsive to the determination, whether the subject more likely has a neurogenerative disease other than Alzheimer's disease.

18. The method according to claim 14, further comprising an act of identifying, responsive to the determination, whether the subject should be categorized in a subgroup of subjects having similar neurophysiology.

19. The method according to claim 14, further comprising an act of identifying, responsive to the determination, whether the subject is predicted to have clinically relevant biomarkers, imaging features, or clinical assessments.

20. The method according to claim 14, further comprising an act of identifying, responsive to the determination, whether the subject is indicated for a therapeutic intervention.

21. The method according to claim 14, further comprising an act of identifying, responsive to the determination, whether the subject is predicted to have a clinical benefit to one or more therapies.

22. The method according to claim 14, further comprising an act of identifying, responsive to the determination, whether the subject is predicted to have an adverse event responsive to receiving one or more therapies.
